(19) United States
(12) Patent Application Publication
(10) Pub. No.: US 2024/0068054 A1
(43) Pub. Date: Feb. 29, 2024

(54) DEVICES AND METHODS FOR COLLECTING AND ANALYZING BIO-AEROSOL SAMPLES

(71) Applicant: 3M Innovative Properties Company, St. Paul, MN (US)

(72) Inventor: Michael Wohl, Bethesda, MD (US)

(21) Appl. No.: 18/272,043

(22) PCT Filed: Jan. 14, 2022
(86) PCT No.: PCT/US2022/012392

§ 371 Date: Jul. 12, 2023

Related U.S. Application Data
(60) Provisional application No. 63/136,723, filed on Jan. 13, 2021.
(60) Provisional application No. 63/148,195, filed on Feb. 11, 2021.
(60) Provisional application No. 63/222,745, filed on Jul. 16, 2021.
(60) Provisional application No. 63/224,242, filed on Jul. 21, 2021.
(60) Provisional application No. 63/237,909, filed on Aug. 27, 2021.
(60) Provisional application No. 63/255,363, filed on Oct. 13, 2021.
(60) Provisional application No. 63/283,075, filed on Nov. 24, 2021.
(60) Provisional application No. 63/287,911, filed on Dec. 9, 2021.

(51) Int. Cl. C12Q 1/70; A61B 5/097; A61B 10/00; C12Q 1/6851

(52) U.S. Cl. CPC C12Q 1/701; A61B 5/097; A61B 10/0045; C12Q 1/6851

(57) ABSTRACT

A bio-aerosol collection device for collecting a bio-aerosol sample comprising: a hollow housing comprising an inlet; an outlet; passaging fluidly coupling the inlet and the outlet; and a capture substrate disposed in the passaging downstream of the inlet toward the outlet whereby the bio-aerosol sample contacts the capture substrate as the bio-aerosol sample flows from the inlet toward the outlet.

### CROSS-REFERENCE TO RELATED APPLICATIONS

This application claims priority to U.S. Application Ser. No. 63/136,723, filed on Jan. 13, 2021, U.S. Application Ser. No. 63/148,195, filed on Feb. 11, 2021, U.S. Application Ser. No. 63/222,745, filed on Jul. 16, 2021, U.S. Application Ser. No. 63/224,242, filed on Jul. 21, 2021, U.S. Application Ser. No. 63/237,909, filed on Aug. 27, 2021, U.S. Application Ser. No. 63/255,363, filed on Oct. 13, 2021, U.S. Application Ser. No. 63/283,075, filed on Nov. 25, 2021, and U.S. Application Ser. No. 63/287,911, filed on Dec. 9, 2021, the disclosures of which are each incorporated by reference in their entireties.

### BACKGROUND OF THE DISCLOSURE

The present disclosure generally relates to medicine. More particularly, the present disclosure is directed to devices and methods for collecting and analyzing bio-aerosol samples.

There is a need in the medical field for devices to collect samples from individuals for testing, analysis and diagnosis. Sample collection can involve invasive methods of sample collection and non-invasive methods of sample collection. Following collection, the sample can require further manipulation to prepare the sample for analysis.

Invasive methods of sample collection include procedures such as surgery and blood draws. Milder forms of invasive methods of sample collection can include swabbing. Invasive methods can cause pain, discomfort, and stress to the patient. Devices used in invasive sample collection methods can also be expensive and require aseptic handling to prevent contamination of the sample. Non-invasive methods of collecting samples can be advantageous over invasive sample collection methods by reducing pain and discomfort in the individual during sample collection.

Both non-invasive sample collection and invasive sample collection can require further handling and processing of the sample for analysis. Sample collection also includes additional handling of the sample after collection. For example, a sample collected using a swab requires transfer of the sample from its place of origin to a vial where a portion of the swab with the collected sample is transferred. Cytological samples such as saliva and sputum can be collected in a sample collection vial and then a portion of the sample is then analyzed following removal of all or a portion of the cytological sample by pipetting. Other liquid samples such as urine and blood are typically collected in a sample collection vial and a portion of the sample is then analyzed by transferring the sample to a separate container. Risk of sample contamination occurs each time the sample is transferred, which can result in inaccurate results.

The collection of tissue samples can require surgery to collect the sample depending on the location of the tissue to be sampled. Samples such as serum samples require the collection of whole blood and then processing of the whole blood to obtain the serum on which an analytic test is performed. Alternative sample types are desirable to reduce or eliminate patient discomfort and complicated methods for sample collection and sample handling.

Accordingly, there exists a need for alternative non-invasive sample collection devices and methods of collecting a sample from an individual. Devices and methods of the present disclosure minimize sample handling can prevent sample contamination. The devices and methods of the present disclosure also permit a patient to collect a sample by expelling air into the device without the need of a medical professional. The collected sample can then be directly transferred to a testing facility from the patient. Other embodiments of the devices and methods of the present disclosure permit the patient to conduct testing on the collected sample.

### BRIEF DESCRIPTION OF THE DISCLOSURE

The present disclosure is generally related to devices and methods for sample collection. In particular, the present disclosure is directed to devices and methods for collecting an air sample from an individual.

In one aspect, the present disclosure is directed to a bio-aerosol collection device for collecting a bio-aerosol sample. The bio-aerosol collection device includes: a hollow housing including an inlet; an outlet; passaging fluidly coupling the inlet and the outlet; and a capture substrate disposed in the passaging downstream of the inlet toward the outlet whereby the bio-aerosol sample contacts the capture substrate as the bio-aerosol sample flows from the inlet toward the outlet.

In one aspect, the present disclosure is directed to a bio-aerosol collection device for collecting a bio-aerosol sample. The bio-aerosol collection device includes: a hollow housing including an inlet; an outlet coupled to an analytical device; passaging fluidly coupling the inlet and the outlet; and a capture substrate disposed in the passaging downstream of the inlet whereby the bio-aerosol sample contacts the capture substrate as the bio-aerosol sample flows from the inlet toward the outlet and extends through the outlet, wherein at least a portion of the capture substrate contacts a sample region of the analytical device.

In one aspect, the present disclosure is directed to a bio-aerosol collection device for collecting a bio-aerosol sample. The bio-aerosol collection device includes: a capture substrate configured to capture an analyte in the bio-aerosol sample; a first bio-aerosol permeable protective layer disposed on a top surface of the capture substrate and covering the top surface of the capture substrate; and a second bio-aerosol permeable protective layer disposed on a bottom surface of the capture substrate and covering the bottom surface of the capture substrate.

In one aspect, the present disclosure is directed to a bio-aerosol collection device for collecting a bio-aerosol sample. The bio-aerosol collection device includes: a capture substrate configured to capture an analyte in the bio-aerosol sample; a first compartment configured to cover a top surface of the capture substrate; a second compartment configured to cover a bottom surface of the capture substrate and comprising an analytical device wherein the analytical device comprises a sample pad in fluid contact with the bottom surface of the capture substrate; wherein the first compartment and the second compartment are configured to be coupled together to form a seal; and wherein upon introducing a buffer onto the capture substrate releases an analyte from the capture substrate into the buffer, wherein the buffer flows to a sample pad of the analytical device.

In another aspect, the present disclosure is directed to a method of collecting and analyzing a bio-aerosol sample, the method comprising: obtaining a bio-aerosol sample from a subject using a bio-aerosol collection device; obtaining a swab sample from the subject; combining the bio-aerosol sample and the swab sample; and analyzing the combined sample.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is an illustration depicting a longitudinal cross-sectional view of an exemplary embodiment of a bio-aerosol collection device.

FIG. 2 is an illustration depicting a longitudinal cross-sectional view of an exemplary embodiment of a bio-aerosol collection device further including a band pass filter and openings (apertures) in the band pass filter and capture substrate.

FIG. 3 is an illustration depicting a longitudinal cross-sectional view of an exemplary embodiment of a bio-aerosol collection device with the inlet and the outlet in closed configurations using caps.

FIG. 4 is an illustration depicting a cross-sectional view of an exemplary embodiment of a bio-aerosol collection device having a capture substrate support with optional lateral flow assay (LFA)/vertical flow assay (VFA) attachment and cap attachment.

FIG. 5 is an illustration depicting a cross-sectional view of an exemplary embodiment of a bio-aerosol collection device with a bottom cap pressure activated dropper and options of introducing a buffer into the bio-aerosol collection device directly or capping the bio-aerosol collection device with a cap having a buffer pack that releases a buffer from the pack upon placement or tightening of the cap.

FIGS. 6A & 6B are illustrations depicting a longitudinal view (FIG. 6A) and longitudinal cross-sectional view (FIG. 6B) of an integrated vial tube sample collection and assay device having a tapered inner tube with a larger inner diameter (D₁) proximal to the inlet tapering to a narrower inner diameter (D2) distal to the inlet (proximal to the collection region). Also illustrated are vents (outlet), an angled collection substrate having a bridging material to direct sample to integrated assay components (e.g., LFA/VFA), and a window allowing a user to observe the assay result.

FIG. 7 is an illustration depicting a longitudinal view of an exemplary embodiment of a bio-aerosol collection device showing the air and reagent flow chamber of the top portion of the device and the capture region detail.

FIG. 8 is an illustration depicting a longitudinal cross-sectional view of an exemplary embodiment of a bio-aerosol collection device showing buffer/reagent components at the surface of the inner wall of the device.

FIG. 9 is an illustration depicting longitudinal cross-sectional views of an integrated bio-aerosol collection and assay device and assay steps. During the collection step, a subject expels air at the device inlet. Internally, the air sample flows toward and through the capture (collection) substrate where an analyte is collected. A buffer is added at the inlet, which flows to the capture substrate. After contacting the capture substrate, collected analyte is transferred via a bridge to an assay component (e.g., sample pad of a LFA/VFA) to begin analysis. A window allows the user to observe the assay results.

FIG. 10 is an illustration depicting a cross-sectional view of an exemplary embodiment of an integrated bio-aerosol collection and assay device having a conical capture substrate supported by an optional conical capture substrate support and in fluid contact with an assay component. The conical configuration focuses sample flow in the collection region.

FIG. 11 is an illustration depicting a longitudinal cross-sectional view of an exemplary embodiment of a bio-aerosol collection device having a conical collection substrate. An assay device (e.g., LFA/VFA) can be an integrated component of the device or the device can be coupled to an assay device. Air flow travels down the inner tube toward the conical capture substrate and is then directed toward the center-point of the capture substrate. An analyte captured by the capture substrate is transferred to the assay device to begin analysis.

FIGS. 12A & 12B are illustrations depicting a longitudinal view (FIG. 12A) and a cross-sectional view (FIG. 12B) of an integrated bio-aerosol collection device with a conical capture substrate coupled to a lateral flow assay (LFA)/vertical flow assay (VFA). The tube portion of the device can also be reversibly coupled to the bio-aerosol collection device housing. The bio-aerosol collection device capture housing can also be reversibly coupled to the LFA/VFA.

FIG. 13 is an illustration depicting a longitudinal cross-sectional view of an exemplary embodiment of a bio-aerosol collection device with a conical capture substrate and coupled to a LFA/VFA. Analyte can be transferred by introducing a buffer using a cap with buffer pack and/or a buffer vial.

FIGS. 14A-14D are illustrations depicting an exemplary embodiment of a bio-aerosol collection device and enlargements of the venting detail (FIG. 14B), the capture substrate detail (FIG. 14C), and a cross-sectional view of the collection substrate/assay component juncture (FIG. 14D).

FIG. 15 is an illustration depicting a cross-sectional view of an exemplary embodiment of an integrated bio-aerosol collection device and LFA/VFA having a “T” shaped design.

FIGS. 16A and 16B are photographs depicting a 3D printed two-part bio-aerosol collection device with a LFA (FIG. 16A) and the capture housing detached from the tube portion) coupled with a LFA.

FIG. 17 is an illustration of an exemplary embodiment of a bio-aerosol collection device depicting a single piece design that allows for the device with the device to be used as a vial by capping the top (inlet) and bottom (outlet). Buffer can be introduced from a buffer vial followed by capping and/or a cap with a buffer pack can be used to cap the device.

FIG. 18 is an illustration of an exemplary embodiment of a bio-aerosol collection device depicting the two-piece construction to lock the capture substrate in place and release the capture substrate by disconnecting the two pieces of the device at a coupling mechanism.

FIGS. 19A-19C are illustrations depicting different mechanisms for removing the capture substrate such as by twisting (FIG. 19A), sliding (FIG. 19B), and pulling (FIG. 19C) whereby the top portion and the bottom portion move independently.

FIGS. 20A-20C are illustrations depicting different mechanisms for transferring the capture substrate out of the bio-aerosol collection device such as by using a probe (e.g., swab or stick) to push the collection substrate out of the device (FIG. 20A), using a movable internal plunger (FIG. 20B), and using a capture substrate with perforations that allow the capture substrate to be torn when pressure is applied to the capture substrate (FIG. 20C).

FIGS. 21A & 21B are illustrations depicting transfer of the capture substrate out of the bio-aerosol collection device where it can be retrieved as it drops out of the device (FIG. 21A) and transferring the capture substrate into a vial that is attached to the device (FIG. 21B).

FIGS. 22A-22C are illustrations depicting elution of capture substrate. In FIG. 22A, the capture substrate remains in place at all times and the patient introduces an air sample (oral/nasal air sample) into the device. In FIG. 22B, a vial is coupled to the bottom of the device. The capture substrate can be entirely or partially dislodged for the elution step to allow the eluent to settle at the bottom. Alternatively, perforations or adhesives attaching the capture substrate to device can be mechanically disrupted with mechanical vibration. Elution buffer can be added and then the top of device is capped. Vigorous mechanical vibration can be applied to elute the capture substrate and gather the elution in the attached vial. In FIG. 22C, the capture substrate remains in the device and buffer is added. The buffer elutes contents of the capture substrate and is collected in the attached vial. The vial is then detached and buffer containing analyte can be transported, stored, and/or analyzed. The vial can also be capped.

FIGS. 23A-23C are illustrations depicting combination of bio-aerosol collection device with swab use. In FIG. 23A, the capture substrate remains in place at all times and the subject blows into device as instructed. In FIG. 23B, a vial is coupled to the bottom of the device. The collection substrate can be entirely or partially dislodged using a swab for the elution step to allow the eluent to settle at the bottom. Elution buffer can be added to the device to simultaneously elute/extract the swab and capture substrate. Alternatively, perforations or adhesives attaching the capture substrate to device can be mechanically disrupted with mechanical vibration. Vigorous mechanical vibration can be applied to elute the swab and capture substrate and gather the elution in the attached vial. In FIG. 19C, the swab and capture substrate remain in the device and elution buffer is added. Elution buffer simultaneously elutes contents of the swab and capture substrate and eluent is collected in the attached vial. The vial is then detached and eluent can be transported, stored, and/or analyzed. The vial can also be capped.

FIG. 24 is an illustration depicting an exemplary embodiment of a bio-aerosol collection device with a funnel (illustrated with optional vents) to accommodate a vial having a smaller size than the bio-aerosol collection device.

FIG. 25 is an illustration depicting an exemplary embodiment of a bio-aerosol collection device with cap for introducing a buffer and integrated assay device (e.g. LFA).

FIG. 26 is an illustration depicting an exemplary embodiment of a bio-aerosol collection device with cap for introducing a buffer and integrated assay device (e.g. LFA) and combination with a swab for analyzing a combined sample obtained by collecting a bio-aerosol sample and a swab sample.

FIG. 27 is an illustration (exploded view) depicting side views and top views of an exemplary embodiment of a bio-aerosol collection device including a top outer layer, a capture substrate layer, and a bottom outer layer.

FIG. 28 is an illustration (exploded view) depicting side views and top views of an exemplary embodiment of a bio-aerosol collection device including a top outer layer, a capture substrate layer, and a bottom outer layer, and further including protective layers (filters) to protect the top and the bottom surfaces of the capture substrate.

FIG. 29 is an illustration depicting a top view of an exemplary embodiment of a bio-aerosol collection device showing the capture region and folds in the outer layers that aid in removing the outer layers to expose the capture substrate that is then processed for analysis.

FIG. 30 is a semi-transparent illustration depicting an exemplary embodiment of a bio-aerosol collection device showing a capture substrate holder that supports the capture substrate against the force of air applied when the user directs a bio-aerosol sample to the collection region and allows for handling the capture substrate without the user directly contacting the capture substrate.

FIG. 31 is a semi-transparent illustration depicting an exemplary embodiment of a bio-aerosol collection device showing outer layers covering the capture substrate layer and only a portion of the capture substrate holder. The device edge that contacts the user's skin can be flat or arcuate.

FIG. 32 is an exploded view (side view and top view) of an exemplary embodiment of a bio-aerosol collection device including an air flow ring/tube to direct a bio-aerosol sample to a capture substrate region and wherein the capture substrate layer includes a capture substrate holder and capture substrate.

FIG. 33 is an exploded view (side view and top view) of an exemplary embodiment of a bio-aerosol collection device. The capture substrate layer includes a capture substrate and a capture substrate holder. The capture substrate holder is releasably connected to a support and configured to be disconnected from the support to remove the capture substrate from a remainder of the bio-aerosol collection device.

FIG. 34 is an exploded view (side view and top view) of an exemplary embodiment of a bio-aerosol collection device. The capture substrate layer includes a tear drop shaped capture substrate and a capture substrate holder. The capture substrate holder is releasably connected to a support and configured to be disconnected from the support to remove the capture substrate from a remainder of the bio-aerosol collection device.

FIG. 35 is an exploded view (side view and top view) of an exemplary embodiment of a bio-aerosol collection device. The capture substrate layer includes a tear drop shaped capture substrate and a capture substrate holder. The capture substrate holder is releasably connected to a support and configured to be disconnected from the support to remove the capture substrate from a remainder of the bio-aerosol collection device. The exemplary embodiment also includes upper and bottom tabs that are located on the top and the bottom surfaces of the capture substrate layer. Protective layers protect the capture substrate from contamination and physical damage and can filter the bio-aerosol sample.

FIG. 36 is an exploded view (side view and top view) of an exemplary embodiment of a bio-aerosol collection device. The outer layers can include folds to aid in the removal (separation) of the outer layers to expose the inner layers and allow the capture substrate to be handled for transport and/or analysis.

FIG. 37 is an exploded view (side view and top view) of an exemplary embodiment of a bio-aerosol collection device showing the edge of the device intended to be fit the user's skin surface in an arcuate shape.

FIG. 38 is an illustration depicting exemplary embodiments of the capture layer of a bio-aerosol collection device.

FIG. 39 is an illustration depicting exemplary embodiments of the capture substrate holder with exemplary embodiments also depicting the capture substrate holder releasably connected to a support and configured to be disconnected from the support.

FIG. 40 is an illustration depicting exemplary embodiments of a bio-aerosol collection device including a “clamshell” component that includes a top portion having a buffer pack and a bottom component that includes an analytical device (LFA/VFA).

FIG. 41 is an illustration depicting semi-transparent images of a top view of the clamshell embodiment and a bottom view of the claim shell embodiment with the capture substrate closed within the claim shell for analysis.

FIG. 42 is an illustration depicting a top view of the clamshell embodiment and a bottom view of the clamshell embodiment with the capture substrate closed within the clamshell for analysis with the capture substrate shown coupled with the support layer and including top and bottom tabs.

FIG. 43 is an illustration depicting a top view of the clamshell embodiment and a bottom view of the clamshell embodiment with the capture substrate closed within the clamshell for analysis with the support removed from the holder.

FIG. 44 is a photographic image of a bio-aerosol collection device.

FIG. 45 are photographic images of a bio-aerosol collection device showing the holder connected with the support and showing the holder (with capture substrate attached) removed from the support.

FIG. 46 is an illustration depicting an exemplary embodiment of a punch design bio-aerosol collection device. Application of force to the top outer layer or bottom outer layer dislodges the capture substrate to allow the capture substrate to be transferred and processed for analysis.

FIG. 47 is an illustration depicting an exemplary embodiment of a punch design bio-aerosol collection device wherein the capture region is configured to be capped.

FIG. 48 is an illustration depicting an exemplary embodiment of a bio-aerosol collection device including an air flow adaptor. The air flow adaptor is configured to receive the bio-aerosol collection device, which advantageously allows different users to direct a bio-aerosol sample to the same bio-aerosol collection device by using a different air flow adaptor.

FIG. 49 is an illustration depicting an exemplary embodiment of a bio-aerosol collection device including an air flow adaptor configured to allow for collection of a bio-aerosol sample from both nostrils of a user.

FIG. 50 is an illustration of an exemplary embodiment of a bio-aerosol collection device including a buffer pack fluidly coupled to a capture substrate that is fluidly coupled to a sample pad of an analytical device.

FIG. 51 is an illustration of an exemplary embodiment of a bio-aerosol collection device whereby the capture substrate is coupled with an analytical device. After bio-aerosol sample collection, the capture substrate is placed into a vial including a reagent buffer that initiates analysis via the analytical device.

FIGS. 52A-52C are illustrations depicting bio-aerosol sample collection (FIG. 52A) and elution of a swab sample (FIG. 52B) that is then used to elute the bio-aerosol sample. The combined swab and bio-aerosol sample is analyzed by the analytical device coupled to the bio-aerosol collection device (FIG. 52C).

FIGS. 53A & 53B are illustrations depicting the steps used to combine a bio-aerosol sample and swab sample in an integrated bio-aerosol collection device. In steps 1 & 2, a bio-aerosol sample (oral and/or nasal) and a swab sample are obtained from the subject. (FIG. 53A). In step 2, the swab is introduced into the bio-aerosol collection device and a buffer is introduced which elutes analyte from the swab and the capture substrate. The analyte is then transferred to the assay device.

FIGS. 54A-54D are illustrations depicting the steps in combining a swab and bio-aerosol sample. In steps 1 & 2, a bio-aerosol sample (oral and/or nasal) and a swab sample are obtained from the subject. (FIG. 54A). In step 3, a buffer is added to a dropper vial (FIG. 54B). In step 4, the swab is used to transfer the capture substrate from the bio-aerosol collection device into the dropper vial (FIG. 54C). In step 5, the swab is removed and the dropper vial is used to transfer a droplet of the combined sample to an analytical device for analyte detection.

FIG. 55 is an illustration depicting combination of a swab sample and a bio-aerosol sample by separately eluting the swab and capture substrate and then combining the swab sample with the bio-aerosol sample to form a combined elution.

FIG. 56 is an illustration depicting combination of a swab sample and a bio-aerosol sample by eluting the bio-aerosol capture substrate and swab in the same vial.

FIG. 57 is an illustration depicting combination of a swab sample and a bio-aerosol sample by first eluting the swab and then using the swab elution to elute the bio-aerosol capture substrate.

FIG. 58 is a graph illustrating that combining samples results in better detection of SARS-CoV2 (adapted from Jarvis and Kelley, Scientific Reports, 11, 9221 (Apr. 28, 2021)).

FIG. 59 is a table summarizing results of an analysis of a nasal air sample collected using an exemplary device embodiment.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Disclosed are devices and methods for collecting bio-aerosol samples from a subject. In some embodiments, the devices are coupled with analytical devices that advantageously allow for the collection and analysis of a bio-aerosol sample alone or in combination with other sample types (e.g., swabs, sputum, lavage, aspirate, etc.). The samples can be collected from a subject by a medical professional or by the subject with or without the aid of a medical professional.

As used herein, “bio-aerosol sample” is used according to its ordinary meaning as understood by those of ordinary skill in the art to mean an airborne collection of biological material. A bio-aerosol sample can include cells, cellular fragments, fungal spores, fungal hyphae, viruses, proteins, nucleic acids, other biological material, and chemicals.

As used herein, “capture substrate” (and/or “collection substrate”) refers to a surface or material that collects material contained in a bio-aerosol sample. The capture substrate is designed to capture (collect and/or trap) an analyte of interest. The pore size of the capture substrate is selected based on the size of the analyte. Generally, the pore size of the capture substrate is smaller than the size of the analyte of interest such that the analyte will not pass through the capture substrate. The capture substrate can also include a range of pore sizes. The capture substrate advantageously concentrates the sample collected such that a minimal amount of reagent is necessary to perform analysis of the sample. The concentrated sample collection function of the capture substrate also increases test sensitivity. The capture substrate can also be designed to release the analyte of interest. For example, the capture substrate can be designed such that an analyte collected by the capture substrate can be eluted from the capture substrate using a buffer that washes the analyte from the capture substrate or a buffer that causes the capture substrate to dissolve and thereby release the analyte. The capture substrate can also be designed to allow the analyte of interest to move within the capture substrate (e.g., from one region of the capture substrate to another region of the capture substrate). The capture substrate can also be configured to allow the analyte of interest to be transferred from the collection substrate to a test substrate. For example, the capture substrate can include pores, channels, grooves, treatments, a mixture of fiber types and materials and the like, through or along which the analyte travels and or that directs movement of the analyte and/or a carrier containing the analyte to move from the capture substrate. For example, an analyte can travel in channels (e.g., microchannels) of the capture substrate to an analytical substrate or another vial.

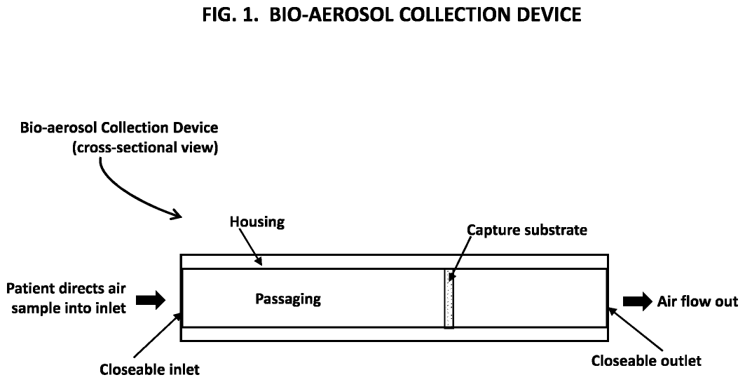

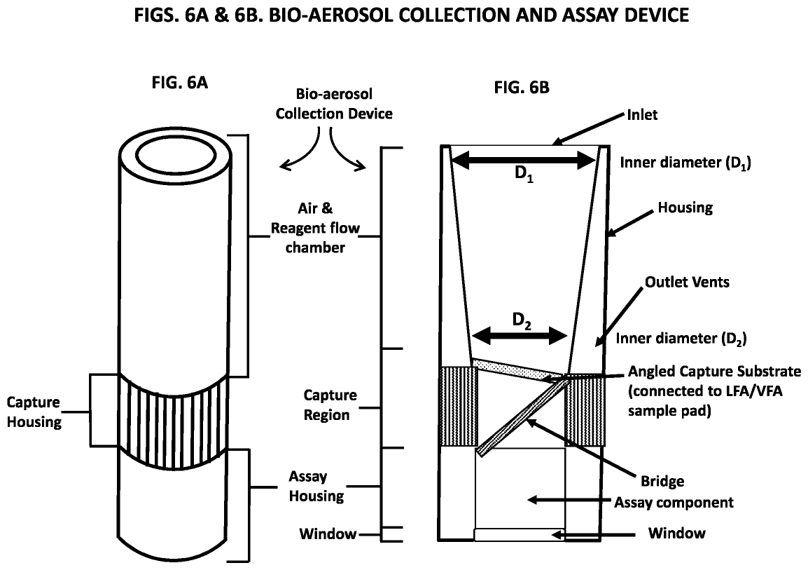

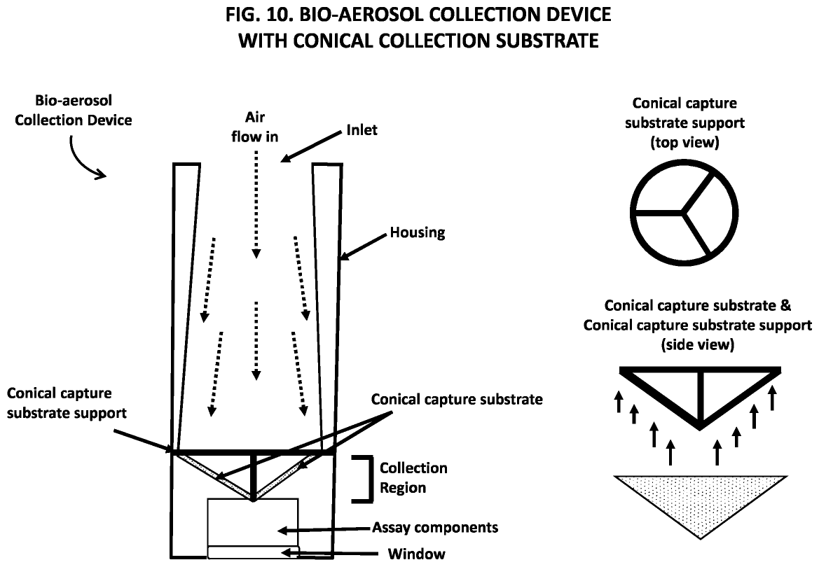

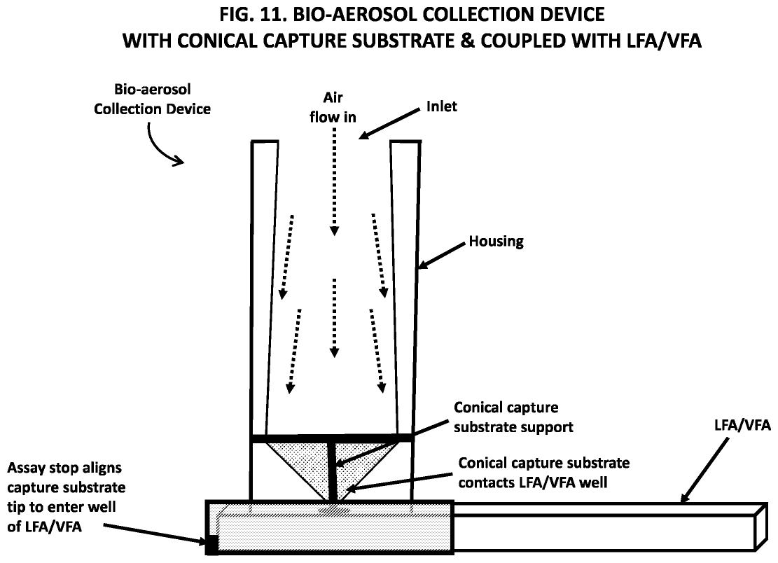

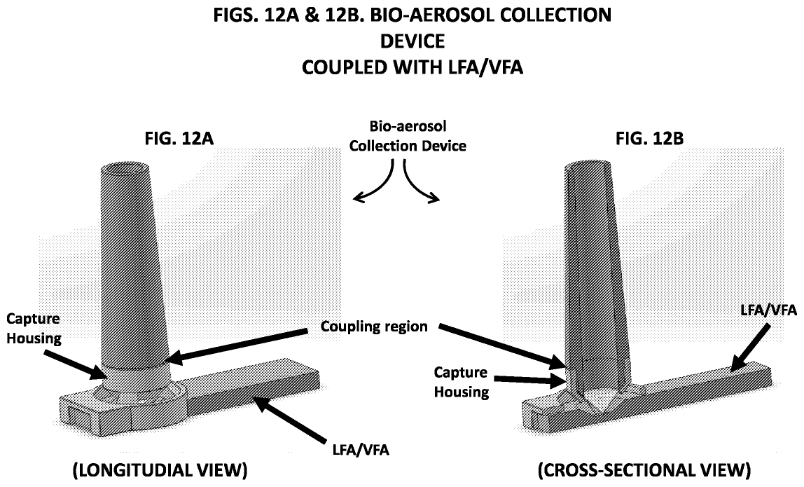

The capture substrate can be in different shapes. Suitable shapes include circular, disc, elliptical, conical, oval, tear drop, square, rectangle, triangle, basket, and the like. Generally, the shape of the capture substrate is similar to the shape of the air flow channel of the device. The shape of the capture substrate can also be similar to the shape of the device designed to hold the capture substrate. The capture substrate can be oriented within the device. For example, as illustrated in FIGS. 1-5, the capture substrate is oriented perpendicular to the airflow. As illustrated in FIGS. 6-9, the capture substrate is oriented at an angle to the airflow. As illustrated in FIGS. 10-15, the capture substrate is oriented perpendicular to the airflow but the shape of the capture substrate is conical, such that portions of the capture substrate are oriented at an angle to the airflow. A conical shaped capture substrate is particularly suitable for focusing the sample material toward the center-point of the substrate. The capture substrate can be shaped similar to the shape of the device designed to hold the capture substrate similar to how a coffee filter fits within a coffee filter holder. The capture substrate can further include pleats, folds, and other arrangements to increase the surface area of the capture substrate.

The capture substrate without the structure of the outer layers is flexible, can fold to adapt to a range of shapes and can easily be placed in a vial. The capture substrate is suitably sized to cover the entire hole (air channel) of the top and bottom outer layers and reduce or prevent a patient's exhaled breath from not passing through capture substrate. The size of the capture substrate is desirably the minimum needed to cover the air channel and hold the capture substrate in place.

The capture substrate can also include perforations. Perforations can be included for removing the capture substrate from other layers. Perforations can also be included to remove a portion of the capture substrate, thus allowing only a portion of the capture substrate to be further tested, for example, while allowing the remaining portion of the capture substrate to be stored for later testing. Testing a smaller portion, rather than the entire capture substrate, may increase sensitivity of the test.

The capture substrate can include “slits” to increase adaptability of the capture substrate to easily conform to a range of vial sizes and for comfort and effectiveness for swabbing a nostril. The slits can be radial slits from the center of the substrate or parallel to the capture substrate holder.

The capture substrate can further include an aperture that allows the user to insert a sample handling device as described herein such as a pipette tip through the capture substrate. The aperture can also function to reduce the pressure of the air flow on the capture substrate such that the capture substrate is not dislodged when the individual directs air into the inlet. While pressure on the capture substrate can be relieved and/or reduced by the aperture, the capture substrate can be made to have a strength that can withstand the pressure created by the individual directing air into the inlet of the device. Further, the capture substrate can be deformed by the air pressure. The material used to make the capture substrate and the method used to fix the capture substrate in position within the housing will prevent the capture substrate from being dislodged while at the same time allow sufficient air flow through the capture substrate.

In certain embodiments, the capture substrate is configured for the additional purpose for use as a swab. Soft bio-aerosol capture substrate material can be used to swab nose (and/or oral) and thus combine a bio-aerosol and nasal (and/or oral) samples thereby augmenting concentration of analyte. For example, all or a portion of the edge of the capture substrate can include a swab material to increase the surface area for swabbing. A capture substrate can include a first layer of a first material to collect analytes in a bio-aerosol sample and a second layer including a swab material.

The capture substrate can be coupled with (or attached to) the capture substrate handle (loop and/or frame), filter layer(s), and outer layers using an adhesive. Adhesive around top and bottom of the air channel can be helpful to make sure no air escapes laterally, but only passes through capture substrate. Suitable adhesives are inert and will not interfere with molecular or antigen testing.

The capture substrate is suitably made with synthetic fibers, natural fibers, and combinations thereof. Fibers used to form the capture substrate include hydrophobic fibers, hydrophilic fibers, and combinations thereof. Hydrophobic fibers include, for example, polylactones, poly(caprolactone), poly (L-lactic acid), poly (glycolic acid), similar co-polymers poly(alkyl acrylate), polybutadiene, polyethylene, polystyrene, polyacrylonitrile, polyethylene (terephthalate), polysulfone, polycarbonate, poly(vinyl chloride), and combinations thereof. Hydrophilic fibers include, for example, linear poly(ethylenimine), cellulose, cellulose acetate and other grafted cellulosics, poly (hydroxyethylmethacrylate), poly (ethyleneoxide), polyvinylpyrrolidone, poly(acrylic acid), poly(ethylene glycol), poly(vinyl alcohol), poly (vinyl acetate), poly(acrylamide), proteins, poly (vinyl pyrrolidone), poly(styrene sulfonate), and combinations thereof. Other suitable fiber materials include, for example, acrylonitrile/butadiene copolymer, cellulose, cellulose acetate, chitosan, collagen, DNA, fibrinogen, fibronectin, nylon, poly(acrylic acid), poly(chloro styrene), poly(dimethyl siloxane), poly(ether imide), poly(ether sulfone), poly(ethyl acrylate), poly(ethyl vinyl acetate), poly(ethyl-co-vinyl acetate), poly(ethylene oxide), poly(ethylene terephthalate), poly(lactic acid-co-glycolic acid), poly(methacrylic acid) salt, poly(methyl methacrylate), poly(methyl styrene), poly(styrene sulfonic acid) salt, poly(styrene sulfonyl fluoride), poly(styrene-co-acrylonitrile), poly(styrene-co-butadiene), poly(styrene-co-divinyl benzene), poly(vinyl acetate), poly(vinyl alcohol), poly(vinyl chloride), poly(vinylidene fluoride), polyacrylamide, polyacrylonitrile, polyamic acid (PAA), polyamide, polyaniline, polybenzimidazole, polycaprolactone, polycarbonate, polydimethylsiloxane-co-polyethyleneoxide, polyetheretherketone, polyethylene, polyethyleneimine, polyimide, polyisoprene, polylactide, polypropylene, polystyrene, polysulfone, polyurethane, polyvinylpyrrolidone, proteins, SEBS copolymer, silk, and styrene/isoprene copolymer. Polymer blends such as, for example, poly(vinylidene fluoride)-blend-poly(methyl methacrylate), polystyrene-blend-poly(vinylmethylether), poly(methyl methacrylate)-blend-poly (ethyleneoxide), poly(hydroxypropyl methacrylate)-blend poly(vinylpyrrolidone), poly(hydroxybutyrate)-blend-poly(ethylene oxide), protein blend-polyethyleneoxide, polylactide-blend-polyvinylpyrrolidone, polystyrene-blend-polyester, polyester-blend-poly(hyroxyethyl methacrylate), poly (ethylene oxide)-blend poly(methyl methacrylate), poly(hydroxystyrene)-blend-poly(ethylene oxide). The fiber materials used to form the capture substrate can be selected to cause the analyte (including a carrier containing the analyte) to travel from one portion of the capture substrate to another portion of the capture substrate. The fiber materials used to form the capture substrate can be selected to cause the analyte (including a carrier containing the analyte) to travel from one portion of the capture substrate out of the capture substrate to an analytical substrate and/or a collection vial.

Another suitable capture substrate can be electret (including thermoelectrets and fibrillated electret film). Electret is a dielectric material having a quasi-permanent electric charge or dipole polarization. Electret can be obtained from commercially available sources. Electret can be prepared by heating and simultaneously exposing a material to an electric field, whereby many dipoles in the material become oriented in a preferred direction. After the heating, the material is “frozen” and is able to keep the position of its electric dipoles for a long period of time. Suitable materials for preparing electrets include, for example, materials can now be used to fabricate thermoelectrets, including organic materials such as ebonite, naphthalene, polymethyl-methacrylate, and many polymers, and inorganic materials such as sulfur, quartz, glasses, steatite, and some ceramics. Electret fibrous membranes are particularly suitable. Suitable polymer electret also includes poly (L-lactic acid) electret and polypropylene. Polyvinylidene fluoride (PVDF)/polytetrafluoroethylene (PTFE) NP electret nanofiber membranes can be formed by electrospinning. Fibrillated electret film van Turnhout (U.S. Pat. No. 3,998,916) is also suitable. Suitable materials to use as the capture substrate include a biosampling material disclosed in Kanzer. U.S. Pat. No. 6,119,691 to Angadjivand discloses an electret filter material. The charged electret interacts with an analyte of interest to capture the analyte. Application of an extraction/elution buffer creates a short in the electric charge causing release of all or substantially all of the analyte from the electret capture substrate material.

Suitable capture substrate materials include those that are dissolvable and/or soluble in a liquid. For example, cellulose acetate nanofibers that are capable of dissolution upon contact with a liquid. It should be understood that the entire capture substrate and/or portions thereof can be dissolvable or soluble. Advantageously, the capture substrate itself can dissolve completely freeing all sample material into the eluent and without requiring a removal process to remove analyte from the capture substrate or to remove any substrate material. Suitably, the capture substrate can be made such that when the capture substrate dissolves, it reacts and stabilizes the sample (e.g., particles, cells, DNA, RNA, and the like) or performs some other service detection. Suitably, the capture substrate may be inert before it is dissolved.

In certain embodiments, the capture substrate is processed to transport, to elute, and/or extract and/or remove an analyte of interest from the capture substrate. In other embodiments, analysis does not require removal or extraction of the analyte from the capture substrate. For example, a capture substrate can be analyzed by adding a reaction solution (e.g., buffer and/or water) to the device that results in a colorimetric reaction indicating the presence or absence of the analyte. In another embodiment, the capture substrate is placed in a reaction solution (e.g., buffer and/or water) whereby the capture substrate dissolves. After dissolving the capture substrate, the analyte to be detected is released into the reaction solution that can be directly tested by addition of other reagents to the device and/or transfer of all or a portion of the solution to another reaction medium such as a vial, tube, membrane, slide, and the like.

The capture substrate can also be protected on all sides by additional layers such as filters and protective layers. The capture substrate can also be released from protective layers. The protective layers and capture substrate can be made of different materials designed for the specific purposes (e.g., protection and sample collection). The capture substrate can be coated with a reagent to retain the analyte load collection. The inner capture substrate and/or protective layer(s) can also be coated with a reagent to stabilize the analyte. The protective layer can be an air-penetrating, touch-protective coating.

As used herein, “buffer” refers to components used for stabilizing, transporting, extracting, eluting, and/or detecting the analyte. For example, analysis reagent can include, for example, buffer components, salts, dNTPs, oligonucleotide primers, polymerases, reverse transcriptases, and combinations thereof. Analysis reagents can suitably be lyophilized, in liquid form, in gels, and combinations thereof. The reagent layer that includes an analysis reagent can be separated by other layers of the collection substrate by a coating to prevent the test reagent from contacting the capture substrate and/or becoming activated until such time that the capture substrate is to be processed for analysis. The analysis reagents can be activated, for example by placing the capture substrate (and/or collection layer with dissolvable layer including a test reagent) in a liquid medium such as a buffer including water, whereby the coating dissolves to release the analysis reagent, which can then also dissolve in the buffer. The coating can be meltable, whereby the temperature can be adjusted such that the coating melts to release the analysis reagent and a mixture can form by which an analyte can be detected. The reagent can also be in the form of microparticles and/or beads that contain the reagents. Suitable reagents include salts, pH buffers, transport media, preservatives, capture reagents, analysis reagents, detection reagents, eluents, antimicrobials such as silver-containing antimicrobial agents and antimicrobial polypeptides, analgesics such as lidocaine, antibiotics such as neomycin, thrombogenic compounds, nitric oxide releasing compounds such as sydnonimines and NO-complexes, bacteriocidal compounds, fungicidal compounds, bacteriostatic compounds, analgesic compounds, other pharmaceutical compounds, adhesives, fragrances, odor absorbing compounds, preservatives, RNAse inhibitors, protease inhibitors, and nucleic acids, including deoxyribonucleic acid, ribonucleic acid, and nucleotide analogs and the like. Other suitable reagents include capture reagents, for example, antibodies that specifically binds an analyte of interest, a ligand that specifically binds an analyte of interest such as, for example, surface molecules, such as sugars, glycoproteins, and the like. Capture reagents can be covalently or noncovalently coupled to the collection substrate by a linker. Any suitable linker can be used such as, for example, organic molecules such as a polymer or copolymer (e.g., a substituted or unsubstituted polyalkylene glycol, such as polyethylene glycol), and/or biological molecules such as bovine serum albumin. During manufacture, a reagent can be introduced to the housing in a liquid medium followed by evaporating the liquid portion of the medium such that the reagent is dried or lyophilized. At a later time, a user can rehydrate the dried reagent by adding a liquid medium where upon rehydration, the reagent is available for its intended purpose. As described herein, analysis reagents can suitably be lyophilized, in liquid form, in gels, and combinations thereof. The analysis reagents can be activated, as described herein by adding a liquid medium such as a buffer including water, whereby the coating dissolves to release the analysis reagent, which can then also dissolve in the buffer. The coating can be meltable, whereby the temperature can be adjusted such that the coating melts to release the reagent and a mixture can form by which an analyte can be detected. The reagent coating can include microparticles and/or beads that contain reagents. Suitable transport media include, for example, viral transport medium (VTM), Amies transport medium, and sterile saline. The transport media can include, for example, RNase inhibitors, DNase inhibitors, protease inhibitors, preservatives, stabilizers, and other reagents for preserving the samples. Once placed in a transport container, the collected and combined samples can be stored until later analysis or processed for analysis. Processing can include lysis, extraction, and other known processing steps to analyze the combined sample for an analyte.

As disclosed herein, an adhesive can be used to “glue” the capture substrate into position within a device (such as glued to a shelf that contacts an edge of the capture substrate and/or to a capture substrate holder. Additionally, or alternatively, the capture substrate can be “locked” into its position within the device where two parts of the device are reversibly coupled and hold the capture substrate (for example, a “clam shell” arrangement). Additionally, or alternatively, the capture substrate can be coupled to a capture substrate holder using melt bonding. Additionally, or alternatively, the capture substrate can be coupled to a capture substrate holder using a fastener such as a rivet, pin, screw, and the like.

In use, the bio-aerosol collection devices are designed to capture or collection analytes contained in bio-aerosol samples. Suitable analytes can be viruses, bacteria, nucleic acids (e.g., DNA and/or RNA), proteins, chemicals, and combinations thereof. As described herein, pore size of the capture substrate is designed such that the analyte of interest is captured by the capture substrate. It should be understood that when the capture substrate includes an aperture to allow a user to insert a device into the device, a certain amount of analyte can flow through the analyte, and thus, will not be captured by the capture substrate. A capture substrate having an aperture will, however, capture an analyte of interest from air that is flowing through the capture substrate.

Analytes include microorganisms, biological molecules, and chemical molecules. Particularly suitable microorganisms to be detected are pathogens. As used herein, “pathogen” refers to microorganisms such as, for example, bacteria, fungi, and viruses. The term “pathogen” also refers to viroids, prions, and proteins.

Suitable analytes are contained in gases and aerosol droplets in air expelled by the user. Suitable analytes include microorganisms, chemicals, proteins, nucleic acids, and combinations thereof. Suitable microorganisms include bacteria and viruses.

Particularly suitable microorganisms include pathogens. The term “pathogen” is used according to its ordinary meaning to refer to bacteria, viruses, and other microorganisms that directly or indirectly cause disease. Exemplary pathogens include, for example, Yersinia, Klebsiella, Providencia, Erwinia, Enterobacter, Salmonella, Serratia, Aerobacter, Escherichia, Pseudomonas, Shigella, Vibrio, Aeromonas, Streptococcus, Staphylococcus, Micrococcus, Moraxella, Bacillus, Clostridium, Corynebacterium, Eberthella, Francisella, Haemophilus, Bacteroides, Listeria, Erysipelothrix, Acinetobacter, Brucella, Pasteurella, Flavobacterium, Fusobacterium, Streptobacillus, Calymmatobacterium, Legionella, Treponema, Borrelia, Leptospira, Actinomyces, Nocardia, Rickettsia, Micrococcus, Mycobacterium, Neisseria, Campylobacter, pathogenic viruses such as, for example, Papilloma viruses, Parvoviruses, Adenoviruses, Herpesviruses, Vaccine virus, Arenaviruses, Coronaviruses (Sars-Cov-2, Coronavirus 229E, Coronavirus HKU1, Coronavirus NL63, Cornavirus OCL43), Rhinoviruses, Respiratory syncytial viruses, Influenza viruses, Picornaviruses, Paramyxoviruses, Reoviruses, Retroviruses, Rhabdoviruses, human immunodeficiency virus (HIV), Taenia, Hymenolepsis, Diphyllobothrium, Echinococcus, Fasciolopsis, Heterophyes, Metagonimus, Clonorchis, Fasciola, Paragonimus, Schistosoma, Enterobius, Trichuris, Ascaris, Ancylostoma, Necator, Wuchereria, Brugi, Loa, Onchocerca, Dracunculus, Naegleria, Acanthamoeba, Plasmodium, Trypanosoma, Leishmania, Toxoplasma, Entamoeba, Giardia, Isospora, Cryptosporidium, Enterocytozoa, Strongyloides, Trichinella, a fungus causing, for example, Ringworm, Histoplasmosis, Blastomycosis, Aspergillosis, Cryptococcosis, Sporotrichosis, Coccidiodomycosis, Paracoccidioidomycosis, Mucomycosis, Candidiasis, Dermatophytosis, Protothecosis, Pityriasis, Mycetoma, Paracoccidiodomycosis, Phaeohphomycosis, Pseudallescheriasis, Trichosporosis, Pneumocystis, Human Metapneumovirus, Human Rhinovirus, Enterovirus, Influenza A, Influenza B, Middle East Respiratory Syndrom Coronavirus (MERS-CoV), Parainfluenza Virus 1, Parainfluenza Virus 2, Parainfluenza Virus 3, Parainfluenza Virus 4, Respiratory Syncytial virus, Bordetella parapertussis, Bordetella pertussis, Chlamydia pneumonia, Mycoplasma pneumoniae, and combinations thereof.

Suitable chemicals include ketones, nicotine, cocaine, opioids, marijuana, benzodiazepines, amphetamines, barbiturates, and the like.

Samples can also be analyzed for proteins, DNA, and RNA.

Samples can be simultaneously analyzed for multiple different analytes (e.g., multiplex assay). For example, the methods of the present disclosure are particularly suitable for detecting and differentiating RNA from SARS-CoV-2, influenza A virus, and influenza B virus in upper or lower respiratory samples in a multiplex assay.

Any method for analyzing the samples of the present disclosure that are known in the art are suitable for analyzing the bio-aerosol sample collected using a bio-aerosol collection device as well as combined samples collected using a bio-aerosol collection device, swabs, lavage, aspirates, and combinations thereof. Suitable methods include, for example, polymerase chain reaction and other DNA and RNA amplification methods (e.g., PCR, RT-PCR, loop-mediated isothermal amplification (LAMP)), immunoassay detection methods such as, for example, Western blot analysis and enzyme linked immunosorbent analysis (ELISA), chromatography such as, for example, HPLC, gas chromatography, capillaripheresis, 2D and 3D gel electrophoresis, mass spectrometry, and combinations thereof.

The sample can be analyzed with devices such as nitrocellulose lateral flow strips that contain one or more capture zones: a control line that detects the presence of all antibodies in the sample and a test line that specifically reacts with an analyte to be detected. The sample can also be analyzed using an antigen test. As known in the art, an antigen test contains antibodies on the test device that will bind to the antigen contained in the sample. In another embodiment, the combined sample can be analyzed with an antibody test. As known in the art, an antibody test contains antigens on the test device that will bind to the antibodies contained in the sample. In both antigen and antibody tests, binding of the antibody and antigen triggers a visible result indicating that the subject is infected.

A typical lateral flow test strip for lateral flow analysis (“LFA”) and vertical flow analysis (“VFA”) includes overlapping membranes mounted on a backing card for better stability and handling. A sample is applied at one end of the strip, on the adsorbent sample pad, which is impregnated with buffer salts and surfactants that make the sample suitable for interaction with the detection system. The sample migrates through the conjugate release pad, which contains antibodies that are specific to the target analyte and are conjugated to colored or fluorescent particles. The sample, together with the conjugated antibody bound to the target analyte, migrates along the strip into the detection zone having specific biological components (e.g., antibodies or antigens) immobilized in lines that react with the analyte bound to the conjugated antibody. Recognition of the sample analyte results in a response on the test line, while a response on the control line indicates the proper liquid flow through the strip. The read-out, represented by the lines appearing with different intensities, can be assessed by eye or using a dedicated reader. Additional test lines of antibodies specific to different analytes can be immobilized in an array format to test multiple analytes simultaneously under the same conditions.

Analysis can be automated using commercially available platforms such as Cobas Amplicor (Roche Molecular Diagnostics, Pleasanton, CA).

Analysis can use point of care platforms and/or offsite high throughput platforms.

The bio-aerosol collection devices of the present disclosure allow for the collection of a bio-aerosol sample provided or obtained from a subject. A subject can direct an air sample through the mouth (orally), through one or both nostrils, and combinations of the mouth and nose. A subject directs an air sample (by blowing, exhaling, breathing, humming, singing, speaking, counting, coughing, snorting, and combinations thereof). The subject can expel air multiple times, for a set time period, and combinations thereof. The air expelled by the subject enters the bio-aerosol sample collection device through an inlet. Air travels to a capture substrate that is positioned within the bio-aerosol collection device where analytes contained in the bio-aerosol sample are captured. The bio-aerosol collection device also includes an outlet (such as vents) that allow air to pass out of the bio-aerosol collection device after passing through the capture substrate. The outlet is configured to reduce pressure in the device such that the position of the capture substrate is not disturbed/disrupted during the bio-aerosol collection process.

In one aspect, the present disclosure is directed to a bio-aerosol collection device for collecting a bio-aerosol sample. The bio-aerosol collection device includes: a hollow housing including an inlet; an outlet; passaging fluidly coupling the inlet and the outlet; and a capture substrate disposed in the passaging downstream of the inlet toward the outlet whereby the bio-aerosol sample contacts the capture substrate as the bio-aerosol sample flows from the inlet toward the outlet.

In one aspect, the present disclosure is directed to a bio-aerosol collection device for collecting a bio-aerosol sample. The bio-aerosol collection device includes: a hollow housing including an inlet; an outlet coupled to an analytical device; passaging fluidly coupling the inlet and the outlet; and a capture substrate disposed in the passaging downstream of the inlet whereby the bio-aerosol sample contacts the capture substrate as the bio-aerosol sample flows from the inlet toward the outlet and extends through the outlet, wherein at least a portion of the capture substrate contacts a sample region of the analytical device.

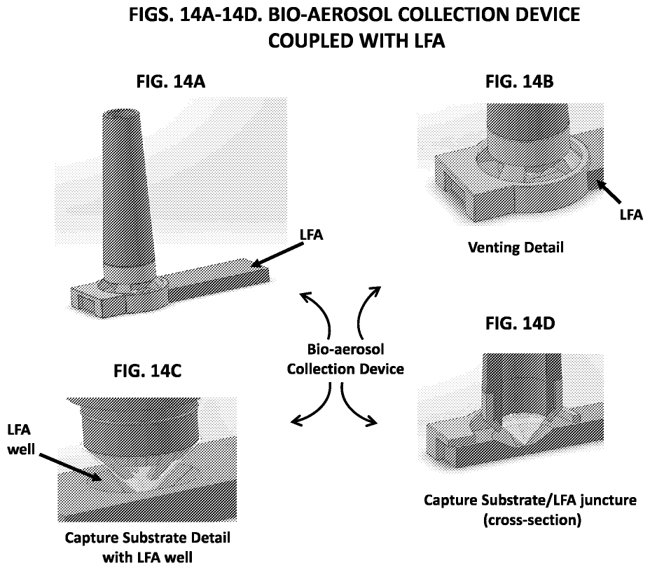

As illustrated in FIG. 1, the capture substrate is disposed between the inlet and the outlet. In certain embodiments, the capture substrate extends through the outlet (see e.g., FIG. 14C).

Suitable capture substrates include those described herein.

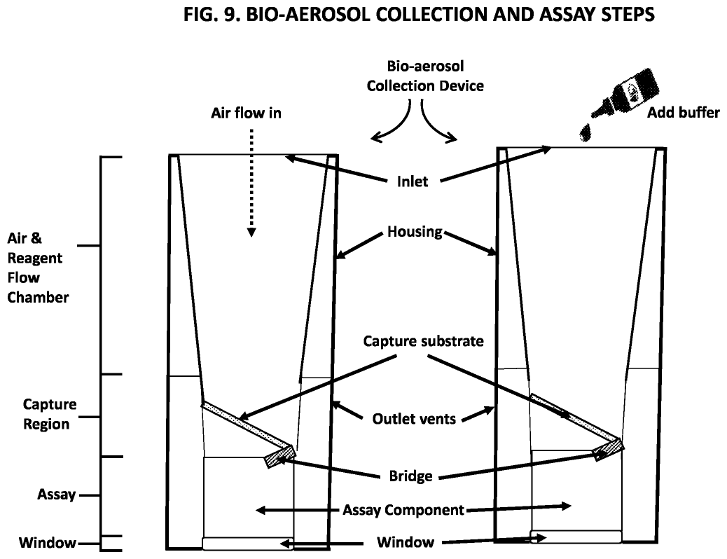

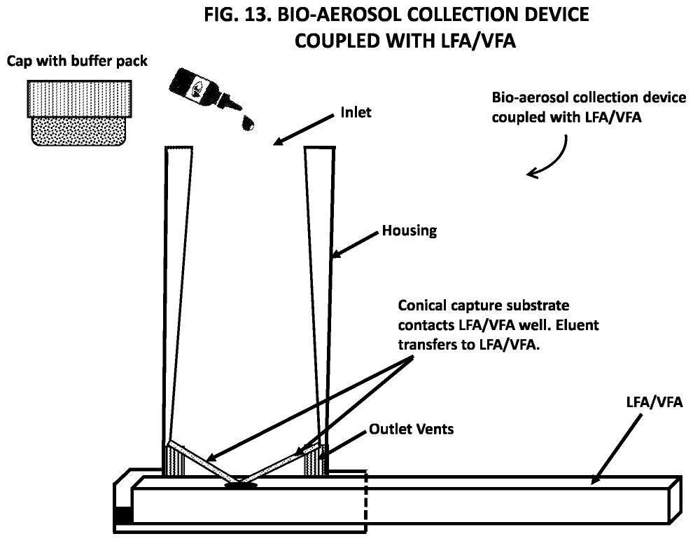

As further described herein, the device is modular meaning that elements can be attached and detached from the device (see for example, FIGS. 9 and 13). For example, the housing of the device can be a single unit or multiple units. Each housing unit can contain one or more components for sample collection, sample processing, and sample analysis. In multi-housing embodiments, housings can be coupled by threading, friction-fittings, and the like. Housings can also be coupled to caps and adaptors to connect the device to sample preparation devices and analytical devices as described herein. In an exemplary embodiment of a modular device, the hollow tube portion can be reversibly coupled to a housing holding the collection substrate.

The hollow housing can be in the form of a cylinder, a tube, a rectangle, an ellipse, and other shapes that allow air directed into the housing through the inlet to pass over or through the collection substrate and exit the housing through the outlet. The term, “hollow” is used according to its ordinary meaning to refer to an empty space or lumen inside the housing member. The empty space allows for directional airflow from the inlet, through the housing, and out the outlet of the device.

The device housing can be made of any material. Particularly suitable materials forming the device housing are compatible with any buffers and reagents useful for detecting an analyte in the sample and that is captured on the collection substrate. Preferably, the housing is made of a material that is transparent, semi-transparent, or translucent to allow the user to see inside the housing. The housing can include a window allowing a user to observe the assay result. The window can also allow the user to transmit the assay result. For example, the user can take a photograph of the assay and transmit the result by electronically/bluetooth sending the photograph image to a receiver. The assay result can also be associated with a scannable code such as a bar code that allows a user to scan the barcode and transmit the test results electronically/Bluetooth.

In one embodiment, a surface of the inner wall of the hollow housing can be hydrophobic. The inner wall can be made hydrophobic by using a hydrophobic material to form the hollow housing. Additionally or alternatively, the inner wall can be made hydrophobic by coating a surface of the inner wall of the hollow housing with a hydrophobic substance. In another embodiment, the inner wall of the hollow housing can be hydrophilic. Additionally or alternatively, the inner wall can be made hydrophilic by coating a surface of the inner wall of the hollow housing with a hydrophilic substance. Additionally or alternatively, a portion of the inner wall can be hydrophobic and another portion of the inner wall can be hydrophilic. The surface of the inner wall can also be treated to reduce surface tension and allow for easier flow of reagents and other liquids along the inner wall.

In one embodiment, a surface of the inner wall is adapted to include a reagent. The surface can be adapted to include a reagent by applying a coating including a reagent to the surface of the inner wall and allowing the coating to dry. The dried coating can be hydrated by adding a buffer or water to the device whereby the buffer or water rehydrates the reagent and can release the reagent from the surface of the inner wall and permit the reagent to interact with the collection substrate.

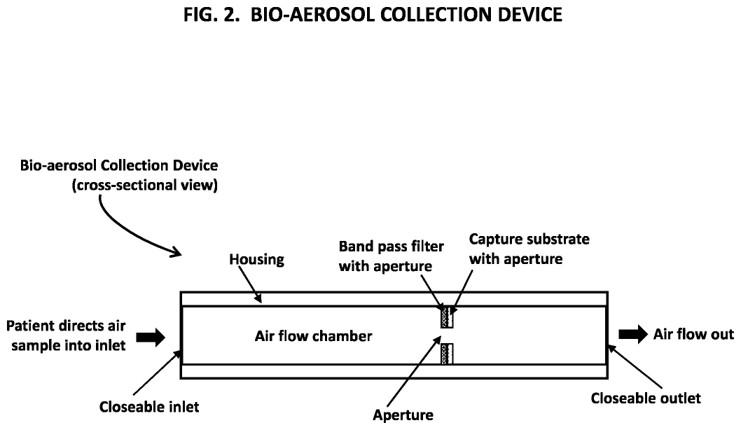

The bio-aerosol collection device can further include a filter (referred to herein as a “band pass filter”), as illustrated in FIG. 2. The band pass filter can capture or trap large particles contained in the bio-aerosol sample. The filter is positioned proximal to the inlet such that particles that are larger in size than the analyte of interest are trapped in the filter material before reaching the collection substrate. Preferred filters include those having a pore size of about 200 μm. Thus, particles contained in the air flow that are larger than 200 μm will be trapped in the filter and particles contained in the air flow that are smaller than the 200 μm pore size can pass through the filter and contact the collection substrate. While a 200 μm pore sized filter is preferred, the filter pore size can be increased or decreased depending on the size of the analyte of interest sought to be captured at the collection substrate.

In one embodiment, the filter is inserted into the same housing containing the capture substrate. In another embodiment, the filter can be contained in a housing that is coupled to the housing containing the capture substrate.

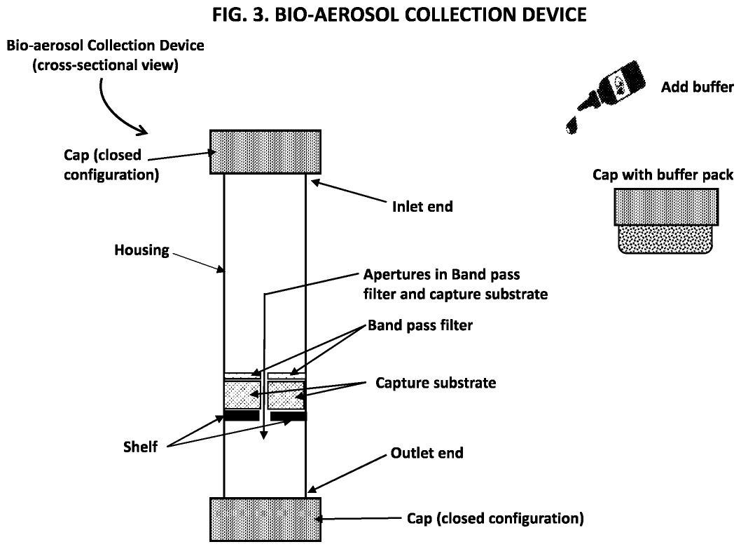

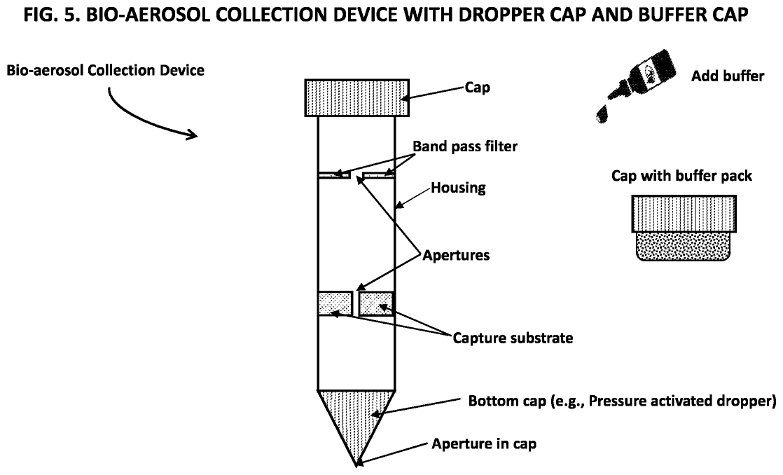

The bio-aerosol collection device can further include a first closure configured to close the inlet, as illustrated in FIG. 3. The bio-aerosol collection device can further include a second closure configured to close the outlet, as illustrated in FIG. 3. Suitable closures include screw caps, snap caps, pressure-fit caps, and the like. Suitable caps can also include caps containing a buffer pack. The buffer pack includes buffer components and reagents as described herein. Upon tightening or pressure applied to the cap, the buffer pack can burst to release a buffer into the device. As illustrated in FIG. 5, the closure can include an aperture (or opening) that permits the device to function as a pressure activated dropper. A liquid (buffer) can be introduced using a cap with buffer pack or from a buffer vial. The device is then closed at the inlet. In certain embodiments, the housing or a portion of the housing of the bio-aerosol collection device is made using a material (soft polymer) that is deformable. A user can apply pressure on the deformable portion of the housing to transfer a drop of liquid through the pressure activated dropper cap. The drop can be transferred to a vial and/or into an analytical device and the remaining liquid can be transported and/or stored.

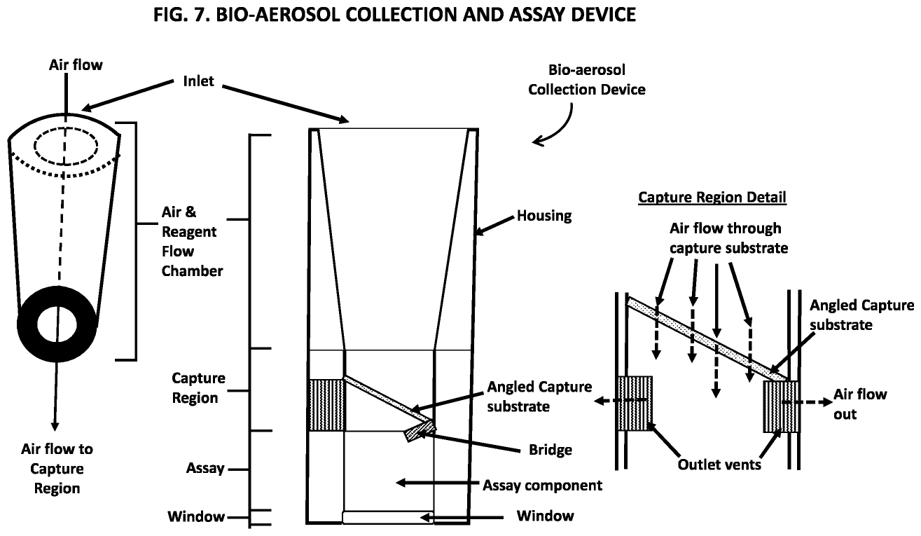

Referring to FIG. 6, the bio-aerosol collection device includes an air and reagent flow chamber having an inner diameter that channels or directs the air sample toward the capture substrate. In some embodiments, the end proximal to the inlet has a larger inner diameter D₁) than the region distal to the inlet (proximal to the capture substrate) (inner diameter D₂). This can focus the air flow as the bio-aerosol sample reaches the capture substrate. In certain embodiments, the inner diameter of the device proximal to the capture substrate is larger than the inner diameter of the device proximal to the inlet (see e.g., FIG. 10). The outlet (such as venting) permits air that has flowed through the capture substrate to pass out of the device (see, FIG. 7). This reduces pressure on the capture substrate that may dislodge or displace the capture substrate. A bridge material in contact with the capture substrate uses capillary action and/or microfluidic channels to transfer buffer containing analyte released from the capture substrate to the assay component to begin the process of analyte detection. The device can further include a window to visualize the results of the analytical assay.

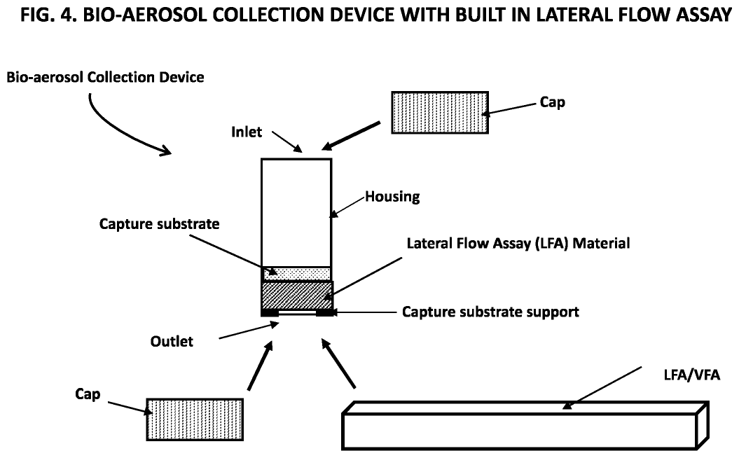

The bio-aerosol collection device can further include a capture substrate support (see e.g., FIGS. 4 and 10). The capture substrate support contacts a portion of the capture substrate to maintain the position of the capture substrate in the device and/or resist the air pressure during collection of the bio-aerosol sample (i.e., when the subject directs air into the device). The support can also be a shelf (see e.g., FIG. 4), a conical (or cone) shape (see e.g., FIG. 10), a basket shape, and other shapes corresponding to the shape of the capture substrate. The capture substrate support can be a spoke system that holds the capture substrate open while not causing a pressure drop.

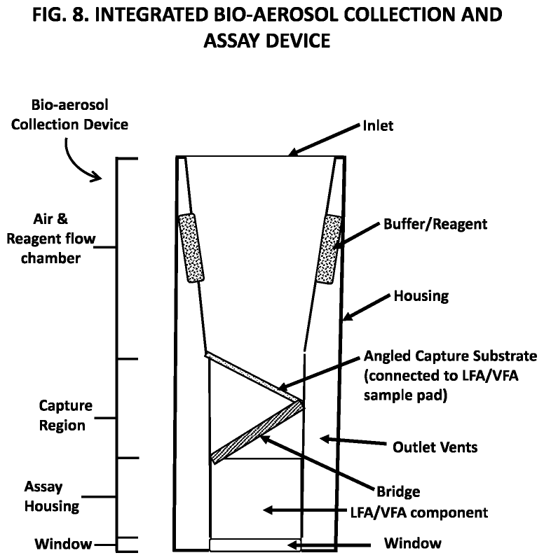

As illustrated in FIG. 8, the device can further include buffer and/or reagent components on an inner surface of the housing. These buffer/reagent components can be in lyophilized form, for example, that are hydrated upon introducing a buffer.

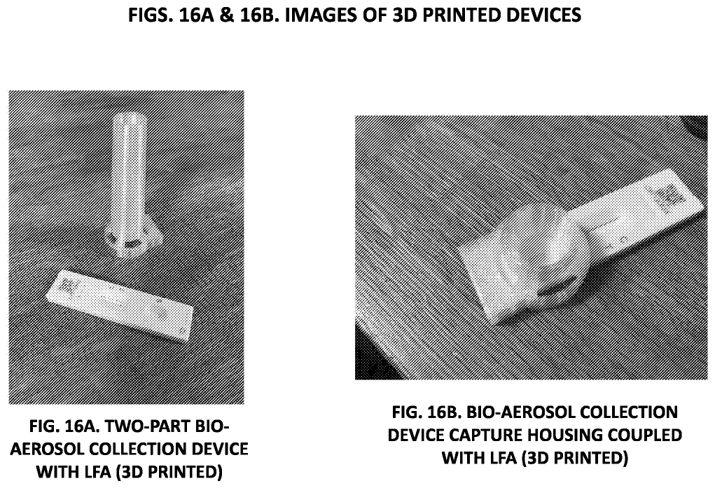

The bio-aerosol collection device can further include an analytical device reversibly coupled to the bio-aerosol collection device (see e.g., FIGS. 16A & 16B). Suitable analytical devices are described herein. As illustrated in FIG. 4, the analytical device (such as a lateral flow assay (LFA)/vertical flow assay (VFA) is coupled to the device. As illustrated in FIGS. 10-12, the capture substrate extends through the outlet and contacts a component of the analytical device (such as a sample pad of a LFA/VFA). A buffer can be introduced to the device to begin the sample analysis, as illustrated in FIG. 13. Generally, the buffer results in elution/extraction of analyte from the capture substrate, which then flows to the analytical device where detection takes place.

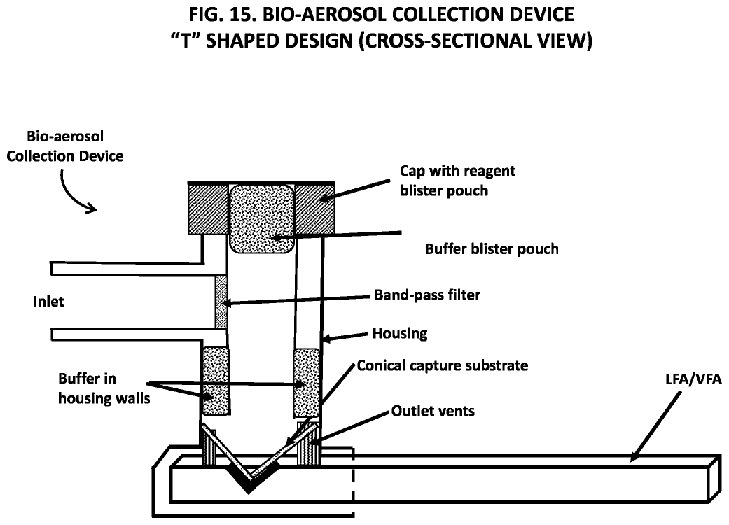

In an exemplary embodiment illustrated in FIG. 15, the bio-aerosol collection device can be formed in a “T-shaped” design such that the bio-aerosol sample initially flows in a direction perpendicular to the capture substrate, which may remove some air pressure on the capture substrate. FIG. 15 also illustrates the use of a band pass filter and a cap with buffer pouch and/or the application of buffer/reagent on the inner wall surface as described herein. A conical shaped capture substrate extends through an outlet to contact a component of the analytical device (e.g., LFA/VFA).

As illustrated in FIGS. 16A & 16B, the device can be modular whereby components of the device can be separated from each other. As shown in FIG. 16A, for example, the housing and capture region can be separated from the analytical device. As shown in FIG. 16B, the housing can be separated from the portion of the device that is coupled to the analytical device.

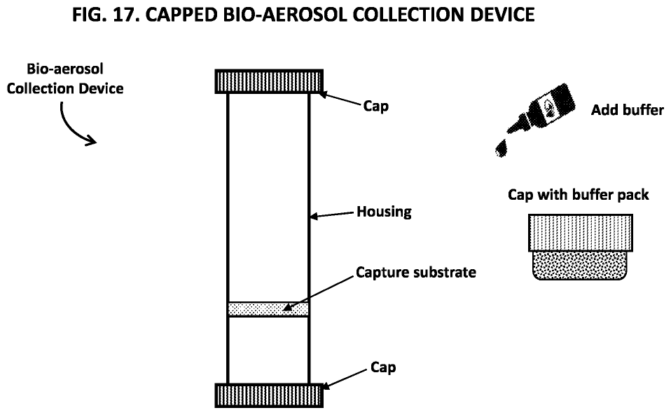

As further illustrated in FIG. 17, the bio-aerosol collection device can be closed using closures such as caps to store and transport the capture device following collection of the bio-aerosol sample. FIG. 17 also illustrates that a buffer can be added directly from a buffer vial and/or using a cap having a buffer pack as described herein.

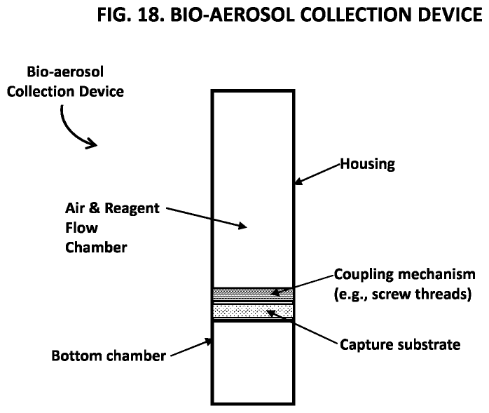

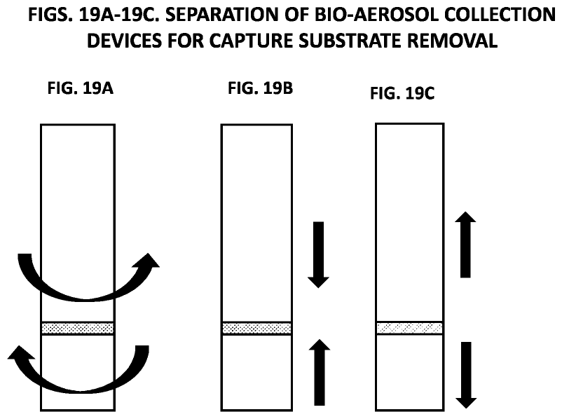

Modularity of the bio-aerosol collection device is further illustrated in FIG. 18. FIG. 18 illustrates using the coupling mechanism such as screw threading between an upper portion (“air & reagent flow chamber”) with the bottom chamber to hold the capture substrate in place within the device housing. As illustrated in FIGS. 19A-19C, the bio-aerosol collection device modular top and bottom chamber components can be separated using a twisting motion, directing the top and bottom chambers together, and/or pulling the top and bottom chambers apart.

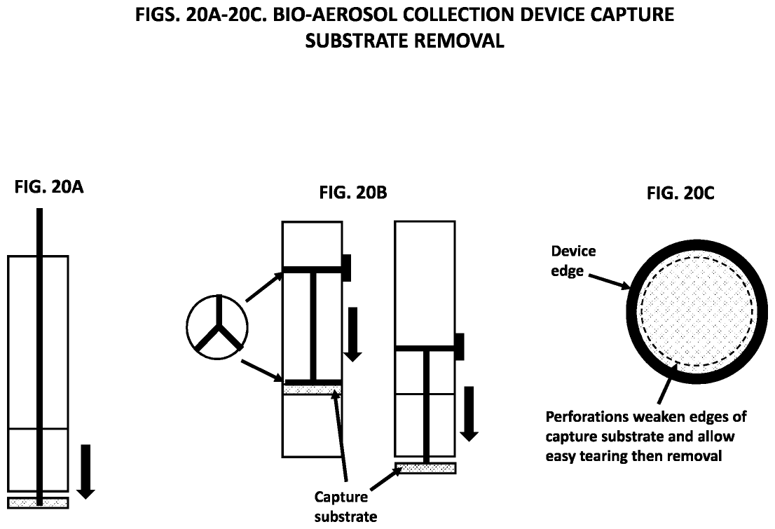

As illustrated in FIG. 20, the capture substrate can be transferred out of the bio-aerosol capture device. Any method to remove the capture substrate is suitable. For example, and as illustrated in FIG. 20A, a stick can be used to dislodge the capture substrate and push it out of the outlet (or inlet). As illustrated in FIG. 20B, an internal plunger device with a slide knob located outside the housing can be pressed to apply pressure that dislodges the capture substrate. To prevent poking a hole in the capture substrate, the transfer stick can have a plunger that applies more dispersed pressure on the capture substrate. As illustrated in FIG. 20C, the capture substrate can include perforations near the outer edge of the capture substrate that allow the capture substrate to be torn from its outer edge where the bulk of the capture substrate can be transferred out of the device.

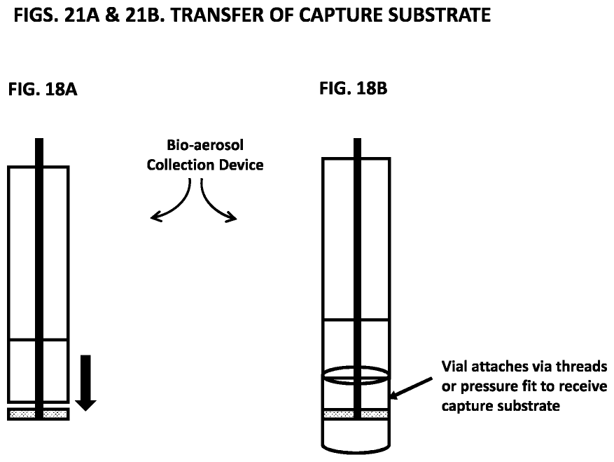

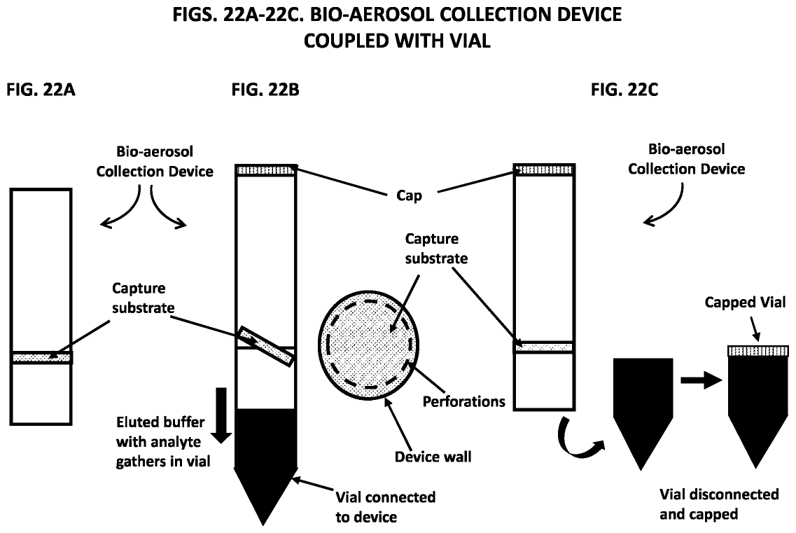

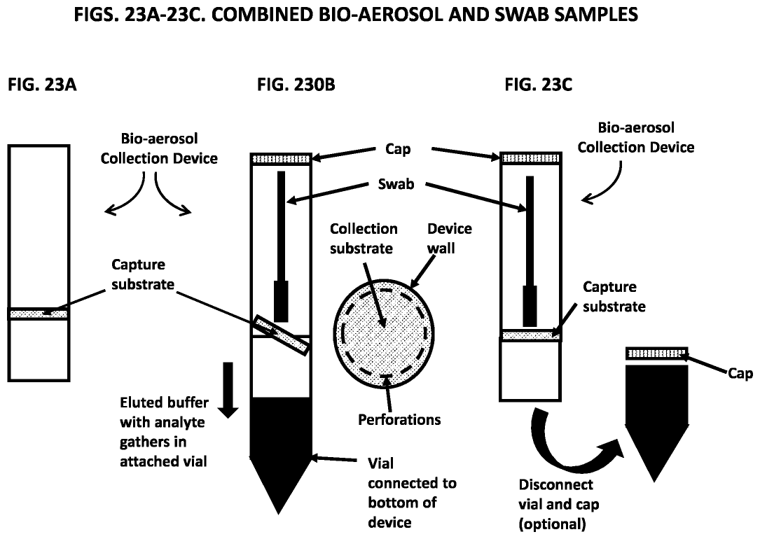

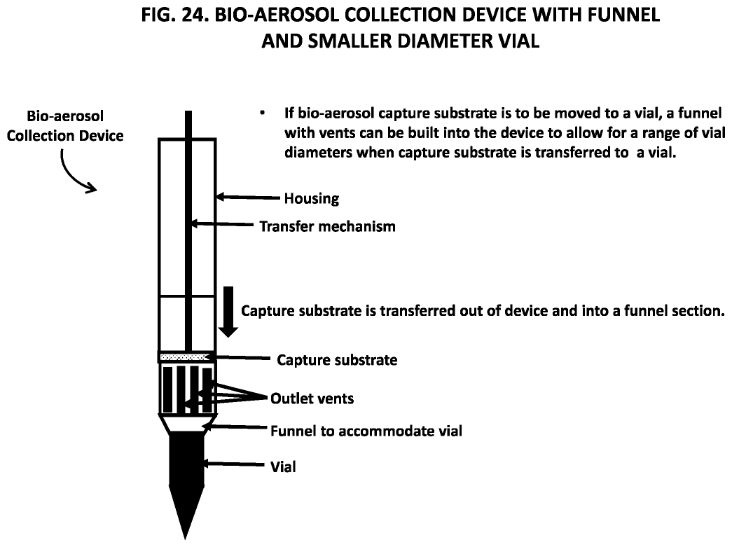

As illustrated in FIGS. 21A and 21B, the capture substrate can be transferred directly into a vial that is reversibly coupled to the device. FIGS. 22-24 illustrate exemplary embodiments of the bio-aerosol collection device configured to reversibly couple to a vial.

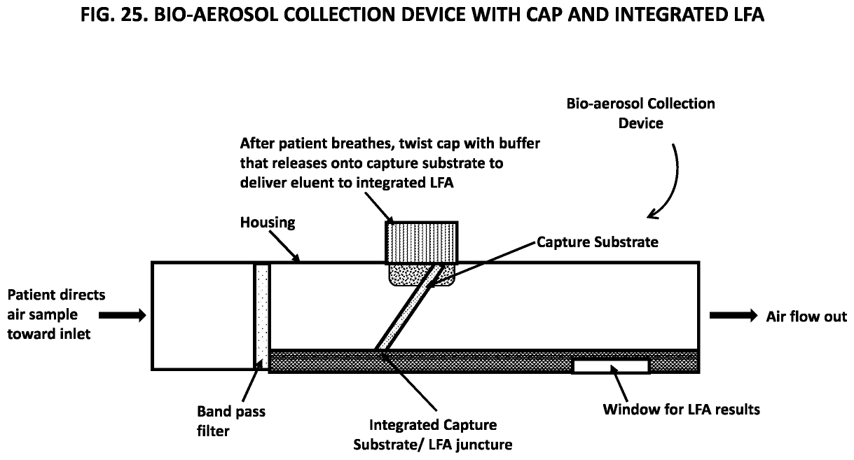

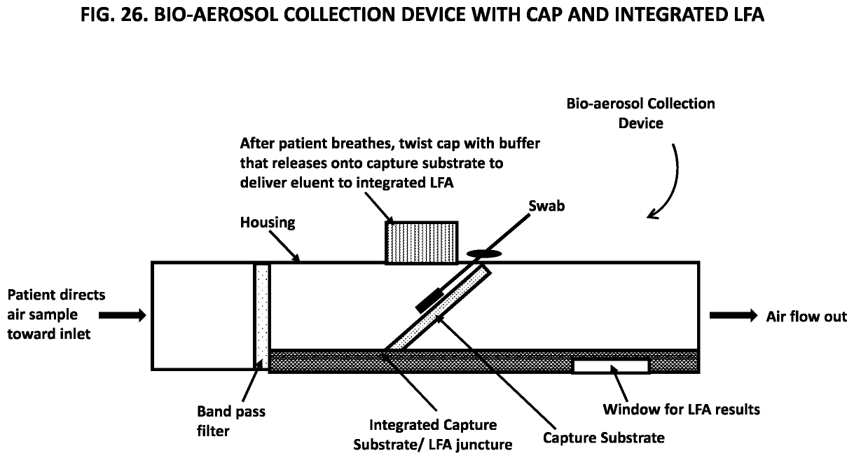

As described herein, one aspect of the present disclosure is directed to a bio-aerosol collection device including an analytical device. FIG. 25 illustrates a bio-aerosol collection device with a cap and integrated analytical device (e.g., LFA). In use, a subject directs an air sample toward the inlet. The air flows through an optional band pass filter and through the capture substrate that captures analyte contained in the air. The device inlet and outlet are closed (not shown) and the cap is tightened, which causes the buffer pack to burst and release the buffer onto the capture substrate. The buffer transfers analyte to the juncture where detection of analyte begins. A window allows the user (or other person) to view the analytical test results. As illustrated in FIG. 26, the device can further include an opening that allows a swab to be introduced into the device. As illustrated in FIG. 26, the swab collection material and capture substrate are desirably placed in proximity such that when the buffer is released, it contacts both the swab and the capture substrate resulting in a combined sample.

In one aspect, the present disclosure is directed to a bio-aerosol collection device for collecting a bio-aerosol sample. The bio-aerosol collection device includes: a capture substrate configured to capture an analyte in the bio-aerosol sample; a first bio-aerosol permeable protective layer disposed on a top surface of the capture substrate and covering the top surface of the capture substrate; and a second bio-aerosol permeable protective layer disposed on a bottom surface of the capture substrate and covering the bottom surface of the capture substrate.

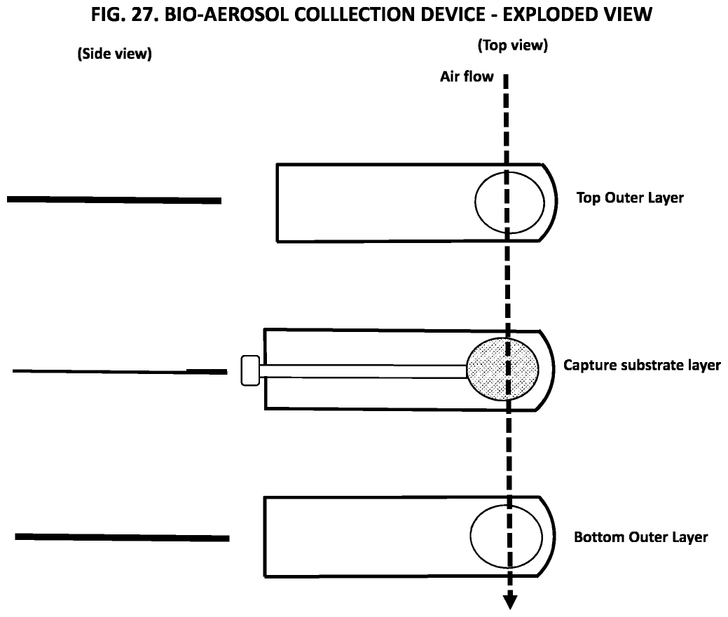

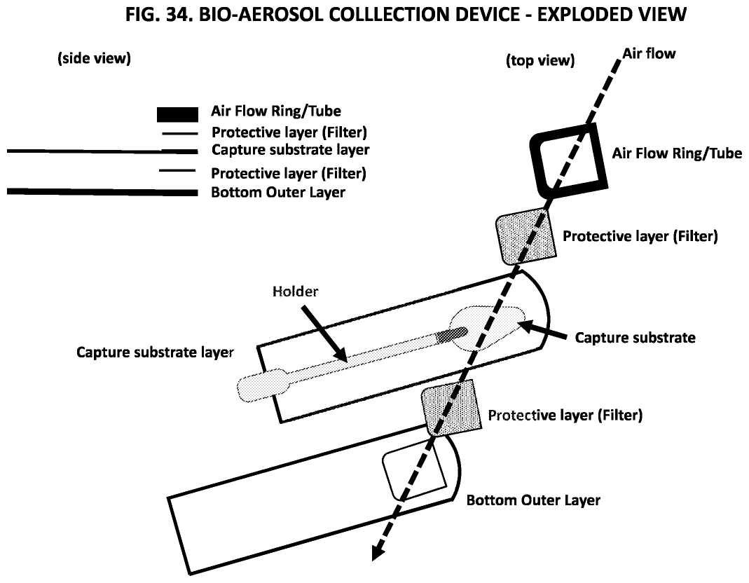

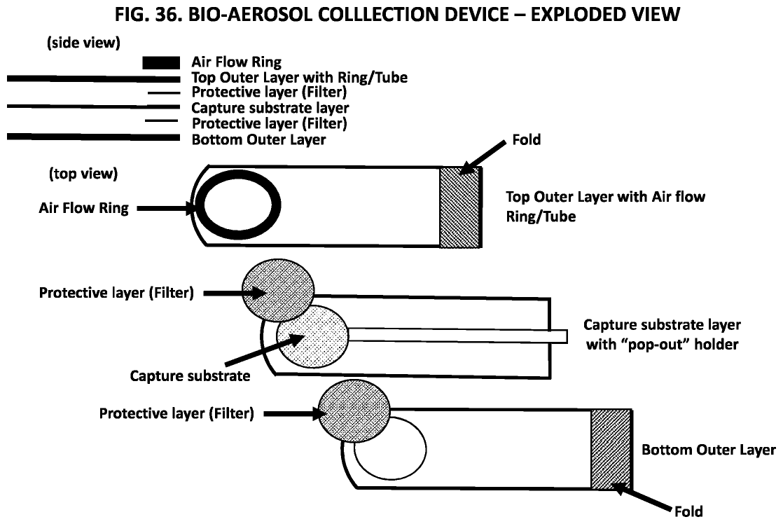

As illustrated in FIG. 27, the bio-aerosol collection device has a top outer layer, a capture substrate layer, and a bottom outer layer. The capture substrate layer includes a capture substrate. As illustrated in FIG. 27, air flows through each of the layers. Analyte in the bio-aerosol sample is captured by the capture substrate as described herein. Suitable capture substrates are described herein. As illustrated in FIGS. 27-37, each of the components of the bio-aerosol collection device together form a passaging. The capture substrate is disposed in the passaging whereby the bio-aerosol sample contacts the capture substrate as the bio-aerosol sample flows. The user directs the bio-aerosol sample toward the capture region, the bio-aerosol sample flows through the top outer layer, through the capture substrate, and through the bottom outer layer. The capture region of the top outer layer and the bottom outer layer can include a protective material (e.g., filter).

In certain embodiments, the bio-aerosol collection device further includes a holder coupled to the capture substrate (illustrated in FIG. 27). In some embodiments, the holder is releasably coupled to the capture substrate. The holder is coupled to the capture substrate using suitable fasteners (e.g., pin, rivet, screw, compression pin, and the like), adhesive, ultrasonic welding, and combinations thereof.

The bio-aerosol collection device can further include a support layer comprising an opening sized and shaped to correspond to the size and shape of the capture substrate. It should be appreciated that the opening and shape of the support layer does not have to be the exact same size and shape of the capture substrate. For example, the size and shape of the capture substrate can be slightly larger than the size and shape of the opening of the support layer, which allows the outer edges of the capture substrate to overlap with the edges of the opening of the support layer and provide a contact surface between the capture substrate and the support. The size and shape of the capture substrate can be slightly smaller than the size and shape of the opening of the support layer such that the capture layer “floats” within the opening of the support layer.

The support layer can further include a holder coupled to the capture substrate whereby the support is releasably connected to the holder and the holder is configured to be disconnected from the support to remove the capture substrate from a remainder of the bio-aerosol collection device.

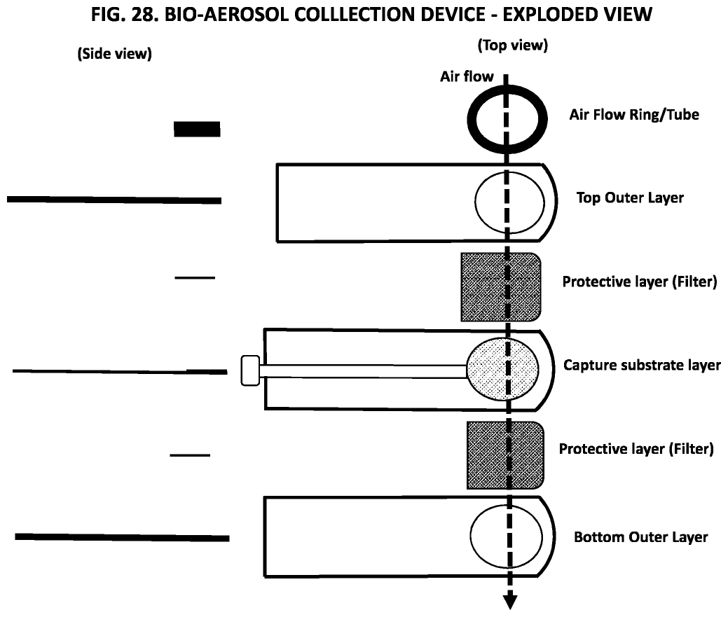

As illustrated in FIG. 28, the bio-aerosol collection device can further include an air flow ring (also referred to herein as “an air flow tube”). The air flow ring/tube directs the user where to position the user's lips, mouth, nostril, or nostrils when preparing to provide a bio-aerosol sample. The air flow ring/tube can be made of any suitable material. The air flow ring/tube can be made of a soft foam to provide comfort and help seal the user's skin surface to the air flow ring/tube.

As illustrated in FIGS. 28 and 32-37, the bio-aerosol collection device can further include a top protective layer and a bottom protective layer. The protective layer(s) can trap (i.e., filter out) unwanted particles contained in the bio-aerosol sample, prevent contamination of the capture substrate by a person inadvertently touching the capture substrate, and prevent physical damage to the capture substrate.

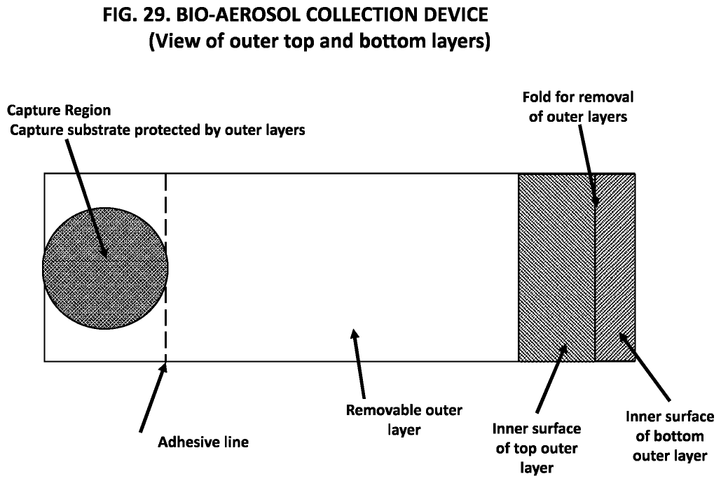

As illustrated in FIG. 29, the outer layers can include folds that assist with removal of the outer layers when it is desirable to expose the capture substrate (e.g., for processing and testing the capture substrate). The outer layers can be peeled apart like an adhesive bandage wrapper. FIG. 29, also illustrates use of an adhesive line proximal to the capture region to provide additional resistance while the outer layers are separated.

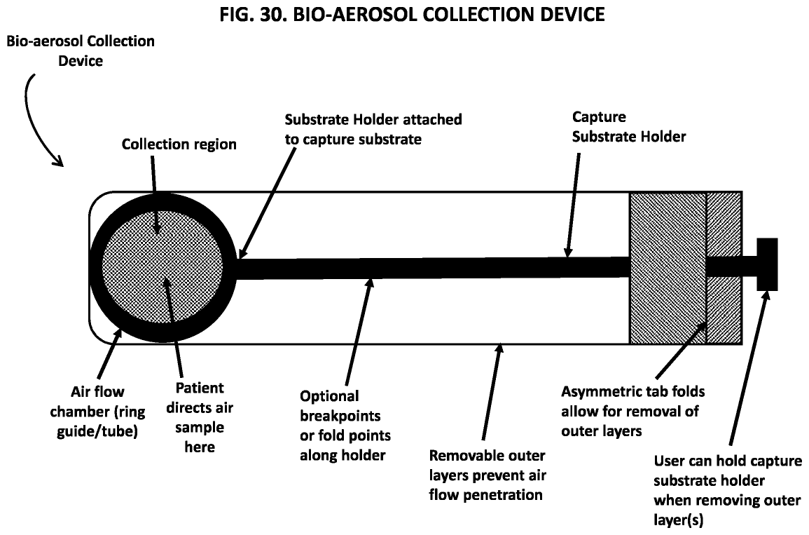

As illustrated in FIG. 30, a portion of the capture substrate holder can extend beyond the outer layers. A user can hold the exposed handle while removing the outer layer (one or both layers). In some embodiments, the handle of the capture substrate holder includes break points that allow the handle to be broken into shorter lengths. This allows the handle length to be shortened to accommodate placement in a vial.

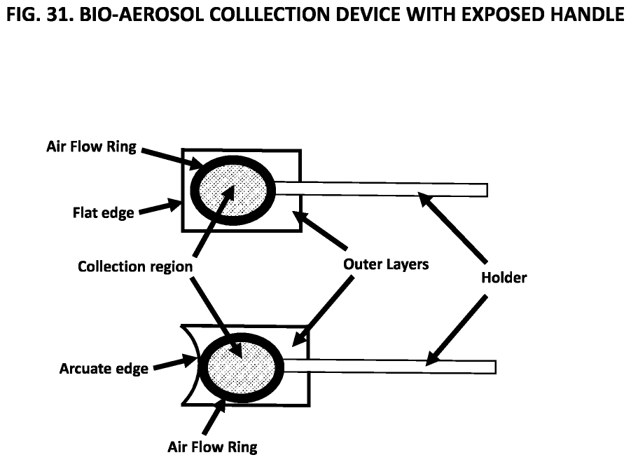

As illustrated in FIG. 31, the outer layers can be sized and/or shaped to be slightly larger than the capture substrate and/or protective layers. This permits use of less materials, while providing support and protection of the capture substrate. FIG. 31 also illustrates that the edge of the device that contacts the user's skin surface can be flat or arcuate, which may create a better seal with the user's skin surface and/or guide the user when positioning the device for bio-aerosol sample collection.

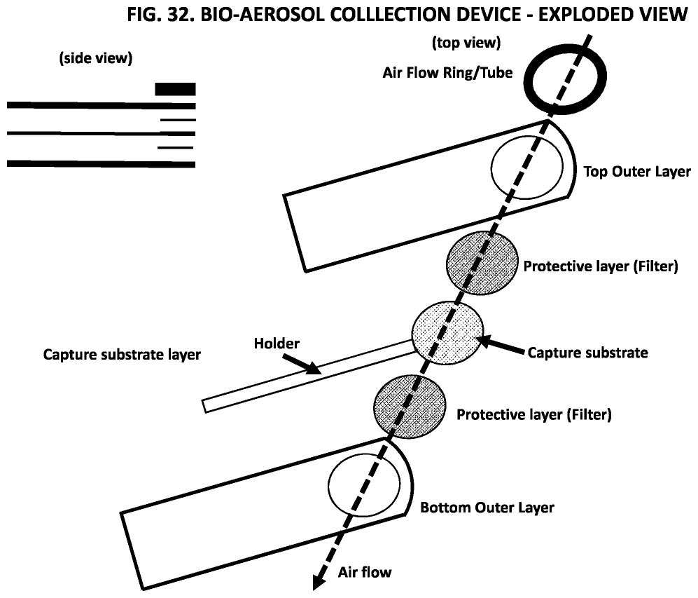

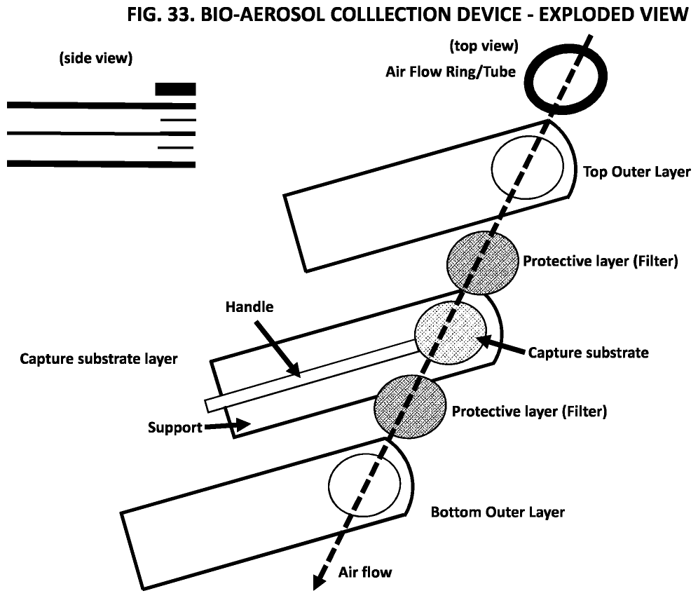

FIG. 32 illustrates one embodiment where the capture substrate layer includes a holder coupled to the capture substrate. FIG. 33 illustrates one embodiment where the capture substrate layer includes a support layer comprising an opening sized and shaped to correspond to the size and shape of the capture substrate and where the support is releasably connected to the holder, the holder configured to be disconnected from the support to remove the capture substrate from a remainder of the bio-aerosol collection device. In some embodiments, the holder is releasably coupled to the capture substrate as described herein. The holder can be broken away from the support material.

The support and capture substrate holder can be semi-rigid or rigid. Suitable semi-rigid and rigid materials are known in the art such as plastics, polymers, metals, and the like. A particularly suitable material for the support and capture substrate holder is high density polyethylene.

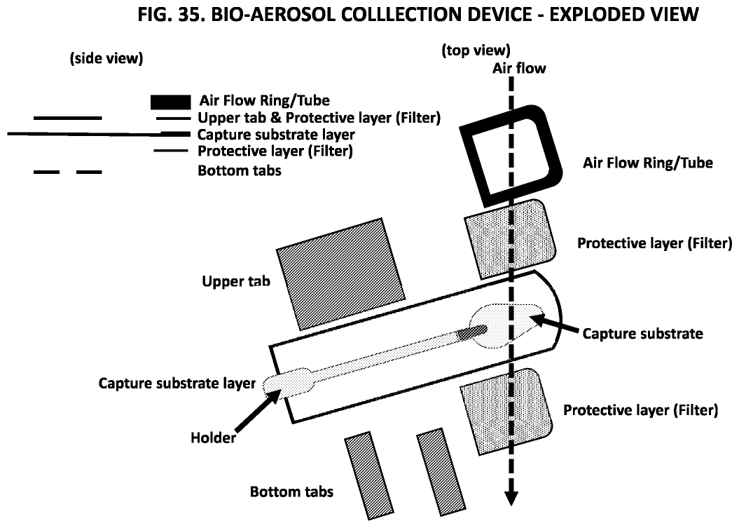

As illustrated in FIG. 35, the holder can be reversibly coupled to the support using tabs such as adhesive material. Use of an upper tab and at least one bottom tab can reversibly couple the capture substrate holder sufficiently to maintain the position of the capture substrate and capture substrate holder. The tabs can also include writing to provide instructions for use or other information.

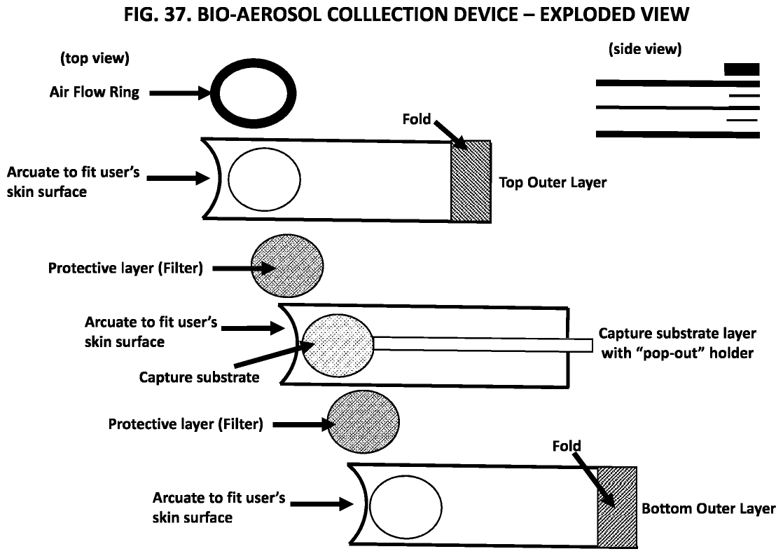

FIG. 37 illustrates a bio-aerosol collection device having an arcuate edge similar to the exemplary embodiment illustrated in FIG. 31, which may create a better seal with the user's skin surface and/or guide the user when positioning the device for bio-aerosol sample collection.

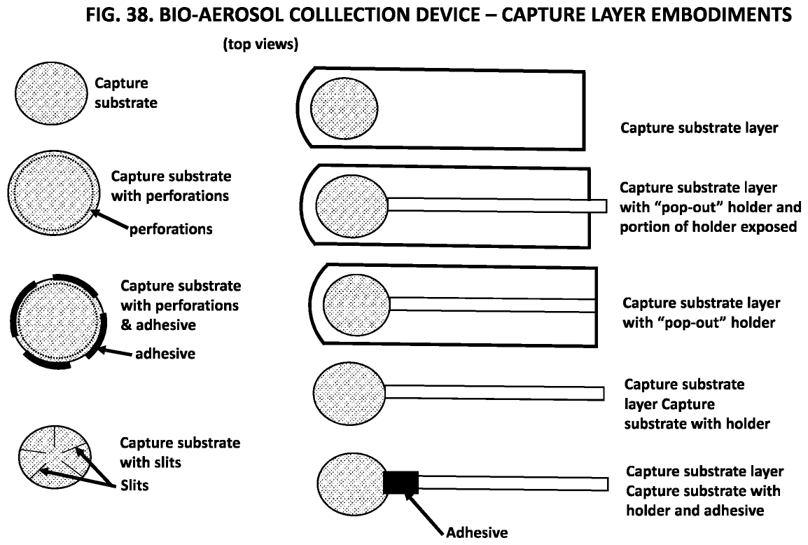

FIG. 38 illustrates exemplary embodiments of the capture layer. The capture layer can be the capture substrate alone. The capture substrate can also include perforations and/or adhesives as described herein. The capture substrate can also include slits (as illustrated in FIG. 38), pleats and folds to increase the surface area of the capture substrate and to allow use of the capture substrate as a swab. The capture substrate can be connected to a holder that can be broken out of a support. As also described, the capture substrate can be permanently or reversibly coupled to the holder as described herein.

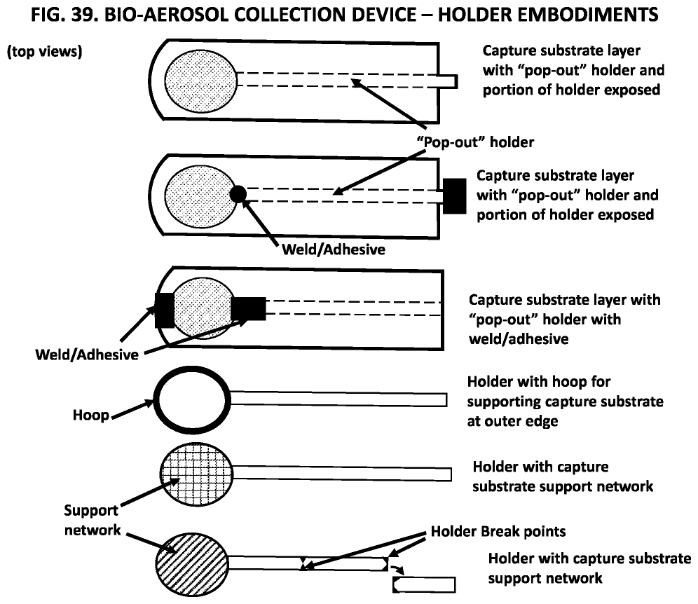

FIG. 39 illustrates exemplary embodiments of the capture holder. As illustrated in FIG. 39, the capture substrate layer includes a support releasably connected to the holder, the holder configured to be disconnected from the support. The holder can be coupled to the capture substrate as described with adhesives, welds, fasteners, and the like. The capture substrate holder can be sized and shaped to support the outer edges of the capture substrate and it can include support networks that support the middle portion of the capture substrate. As also described and illustrated in FIG. 39, the holder includes break points that allow the length of the holder to be reduced (shortened).

In one aspect, the present disclosure is directed to a bio-aerosol collection device for collecting a bio-aerosol sample. The bio-aerosol collection device includes: a capture substrate configured to capture an analyte in the bio-aerosol sample; a first compartment configured to cover a top surface of the capture substrate; a second compartment configured to cover a bottom surface of the capture substrate and comprising an analytical device wherein the analytical device comprises a sample pad in fluid contact with the bottom surface of the capture substrate; wherein the first compartment and the second compartment are configured to be coupled together to form a seal; and wherein upon introducing a buffer onto the capture substrate releases an analyte from the capture substrate into the buffer, wherein the buffer flows to a sample pad of the analytical device.

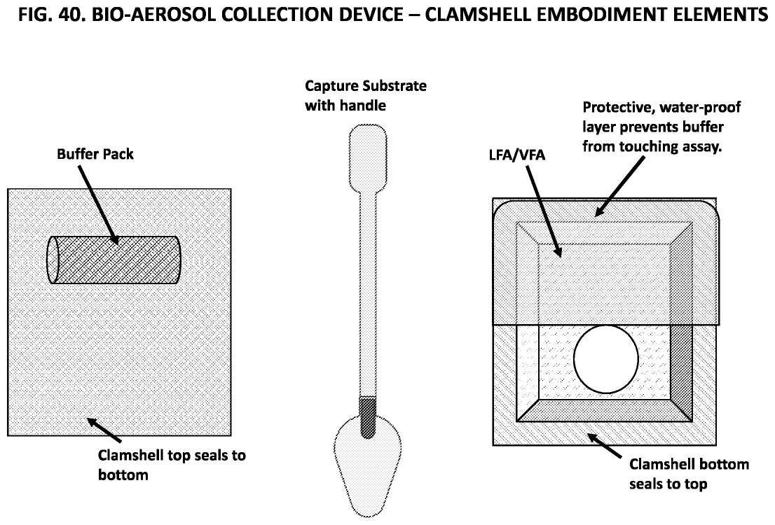

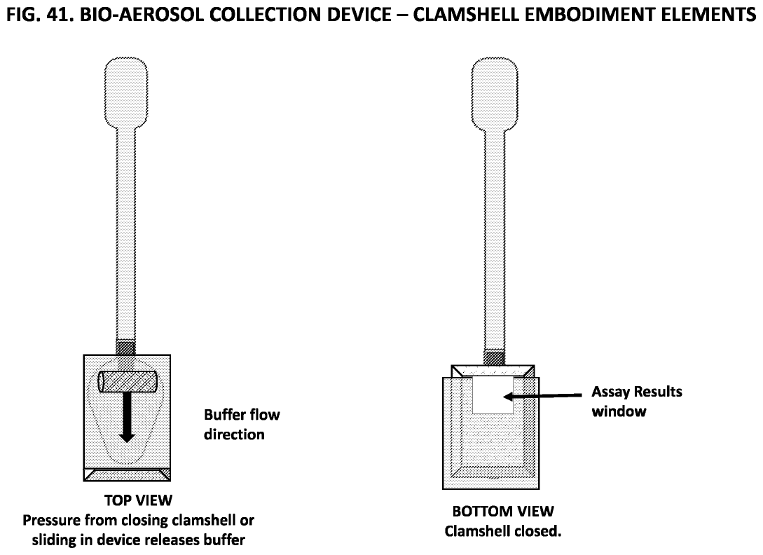

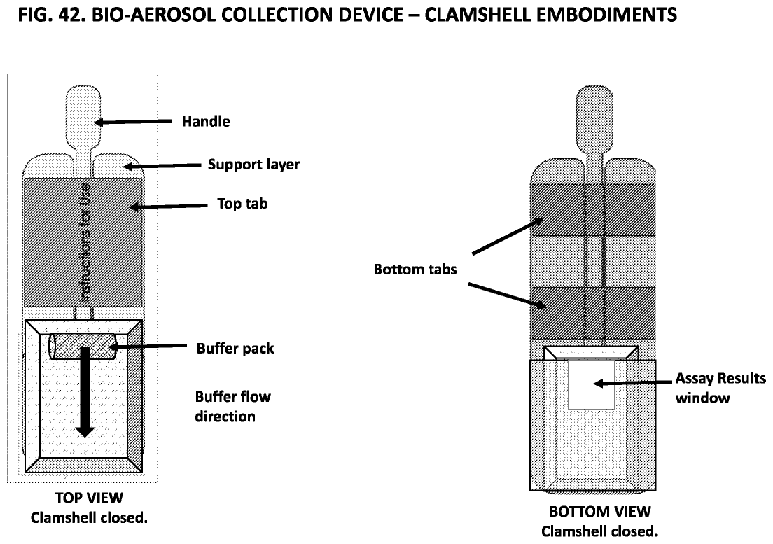

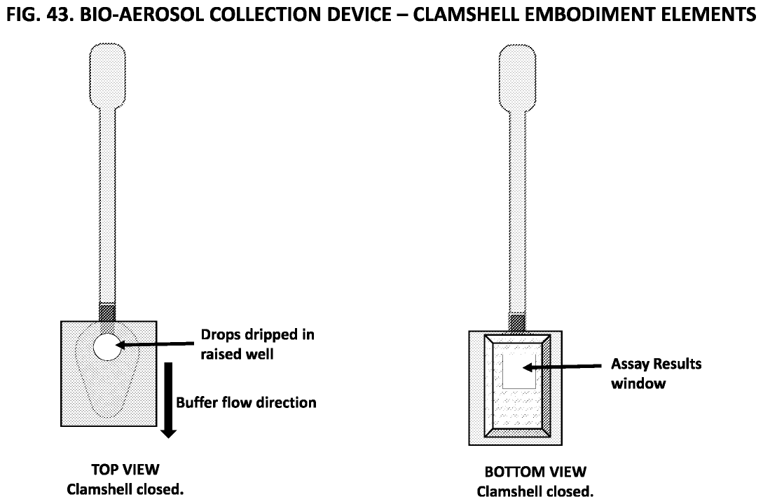

As illustrated in FIG. 40, the top compartment and bottom compartment are in the form of a “clamshell” that encloses the capture substrate. When closed, the top compartment and bottom compartment form a fluid-tight seal. In certain embodiments, the top compartment and the bottom compartment can be coupled using a hinge. In certain embodiments, the top compartment and the bottom compartment exist as separate components. FIG. 40 also illustrates an exemplary embodiment whereby the top compartment includes a buffer pack as described herein that releases a buffer when pressure is placed to couple the top compartment and bottom compartment. A portion of the capture substrate holder can also contact the buffer pack to aid in release of the buffer. The bottom compartment can include additional features that contact the buffer pack to aid in the release of the buffer when the top and bottom compartments are closed. In some embodiments, the bottom compartment includes an assay device such as LFA/VFA. A bridge material can contact the smaller end of capture substrate and sample pad of VFA/LFA to transfer the sample to the LFA/VFA. FIG. 41 illustrates the device in a closed configuration in a top view and a bottom view of the device. FIG. 42 illustrates an exemplary embodiment of the device that includes a support and tabs. FIG. 43 illustrates an exemplary embodiment of a device wherein the top compartment includes a well that permits a buffer to be introduced to the capture substrate through the top compartment.

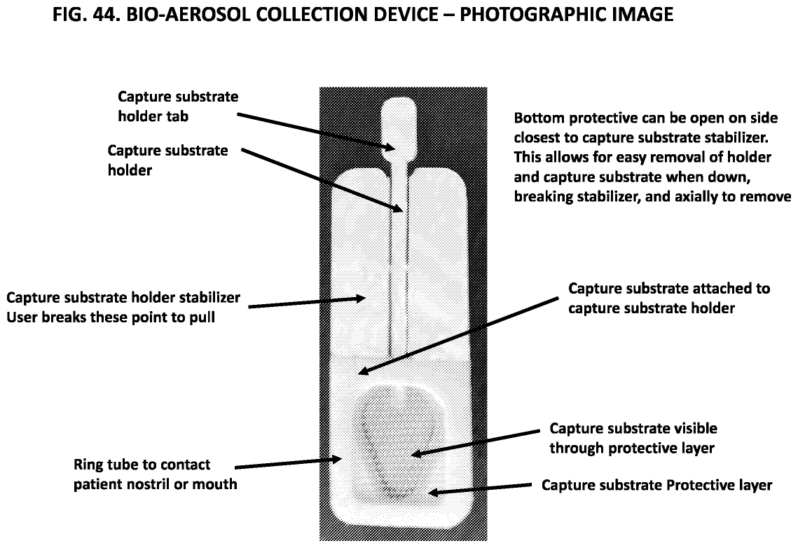

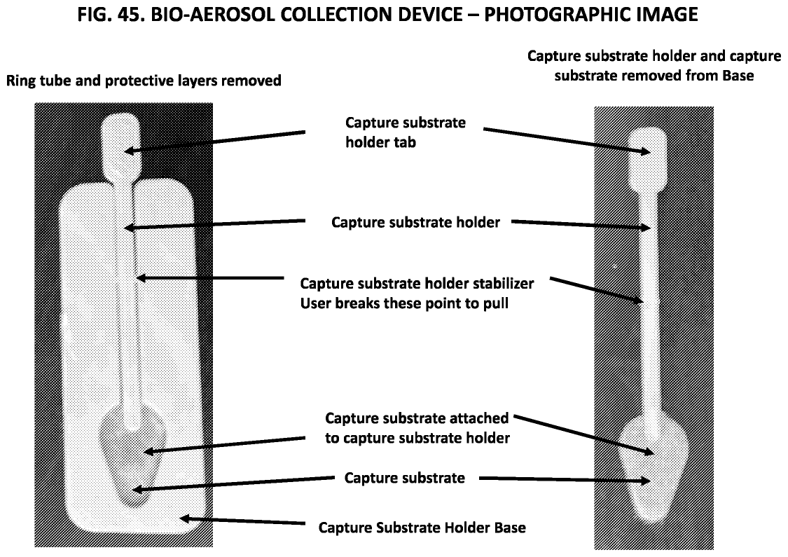

FIGS. 44 and 45 are photographic images of an exemplary embodiments of the bio-aerosol collection device.

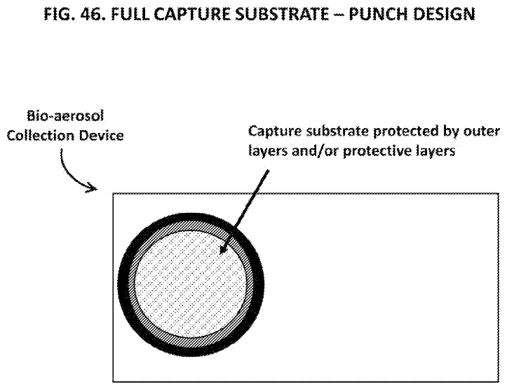

FIG. 46 is an illustration of an exemplary embodiment of a bio-aerosol collection device in a punch design. A hole diameter in bottom outer layer can be smaller than hole diameter in top outer layer. The top hole diameter can accommodate geometry of patient's mouth and/or nostril(s), while bottom hole diameter can be smaller than vial diameter in which to transfer inner collection media. The capture substrate is fully perforated or friction held in device. The bottom protective layer and top protective layer are nearly completely perforated. When pressure is applied, the top and bottom protective layers release but remain attached to outer layers. The user can push the top outer layer down through the collection region to completely dislodge the capture substrate freeing it to be transferred into a vial. The top and bottom outer layers and protective layers can remain hinged attached to the device.

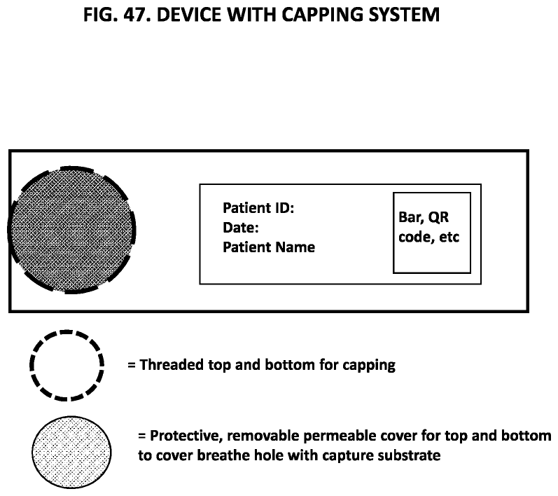

FIG. 47 is an illustration of an exemplary embodiment of a bio-aerosol collection device having a capping system. The top and bottom layers have threading or friction points that engage caps. An optional protective layer for one or both of the top and bottom layer can be positioned over the capture substrate when the user is introducing the bio-aerosol sample. FIG. 47 also illustrates that writing can be applied to the device.

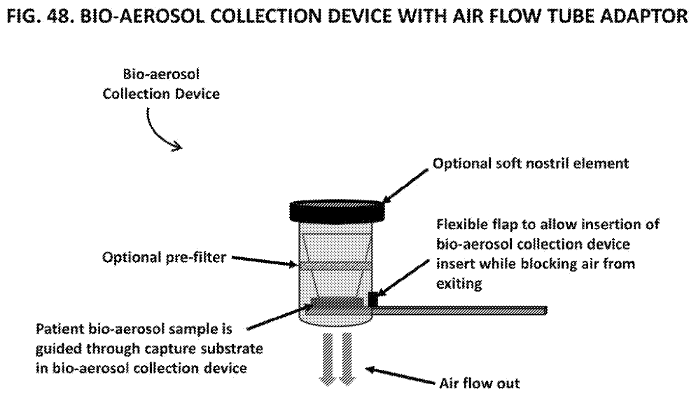

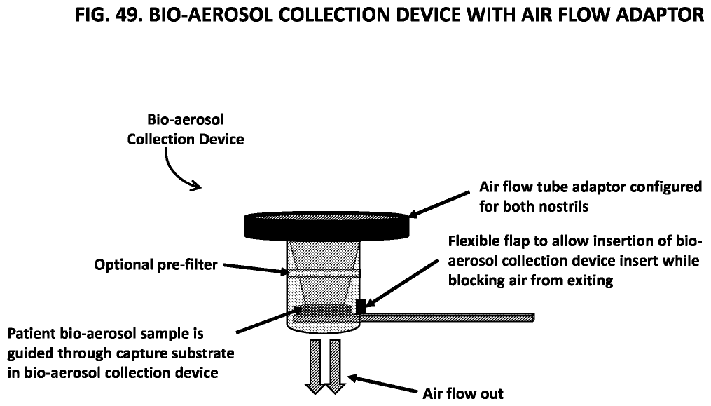

FIGS. 48 and 49 are illustrations of an exemplary embodiment of a bio-aerosol collection device having an air flow tube adaptor. The air flow adaptor includes an insertion opening to receive the bio-aerosol collection device. An optional soft material can be included on the adaptor for comfort. This device is particularly useful for collecting bio-aerosol samples from multiple users using the same bio-aerosol collection device. In use, each user has their own adaptor, but a single bio-aerosol capture device is used by all of the users. For instance, a family of 5 can blow into the same sample collection device to be tested. FIG. 49 shows an air flow adaptor configured for both nostrils of a user.

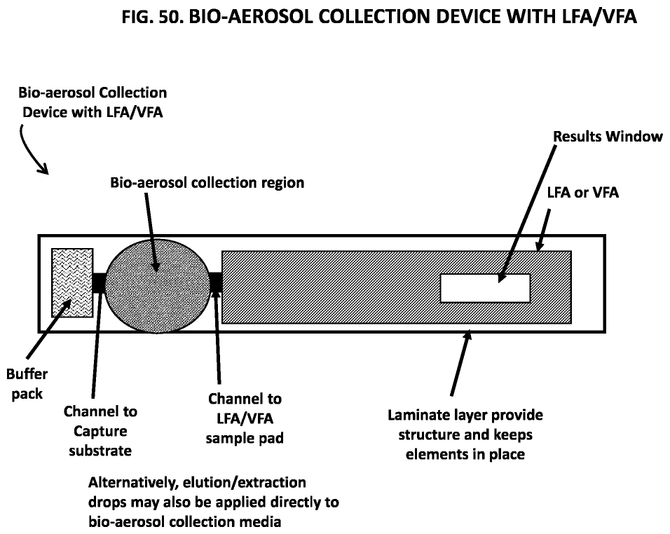

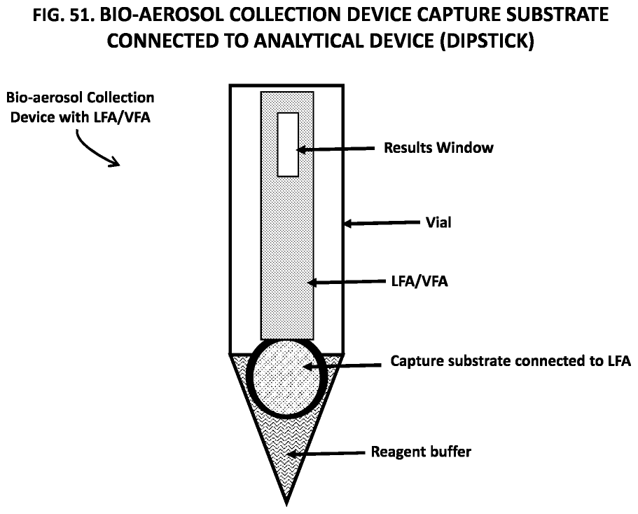

In an exemplary embodiment depicted in FIG. 50, the bio-aerosol collection device includes a buffer pack, a channel fluidly coupling the buffer pack to a capture substrate, and a second channel fluidly coupling the capture substrate to an analytical device. FIG. 51 is an illustration of an exemplary bio-aerosol collection device coupled with an analytical device (LFA/VFA) and transferred to a vial containing a reagent buffer that contacts the capture substrate to initiate analyte detection.

In another aspect, the present disclosure is directed to a method of collecting and analyzing a bio-aerosol sample, the method comprising: obtaining a bio-aerosol sample from a subject using a bio-aerosol collection device; obtaining a swab sample from the subject; combining the bio-aerosol sample and the swab sample; and analyzing the combined sample.

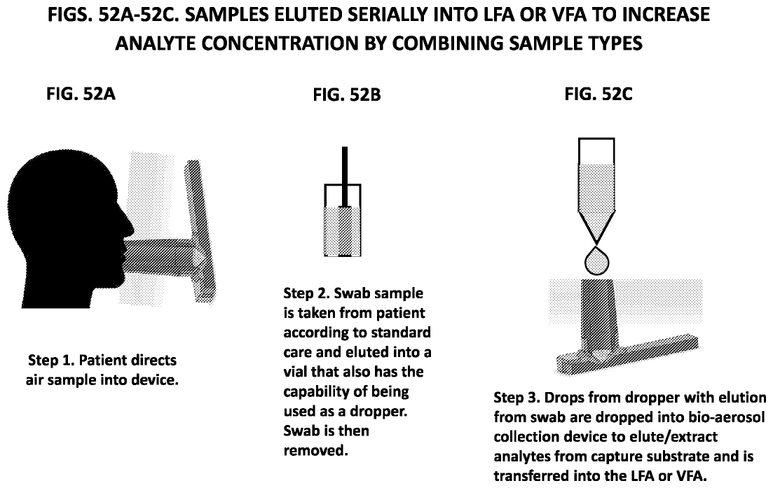

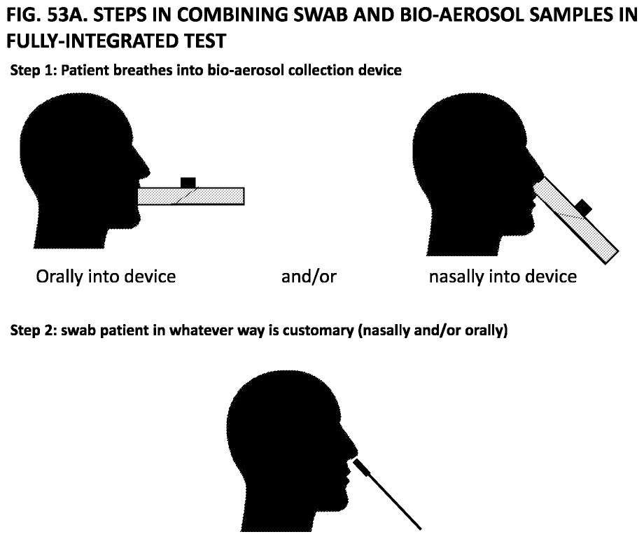

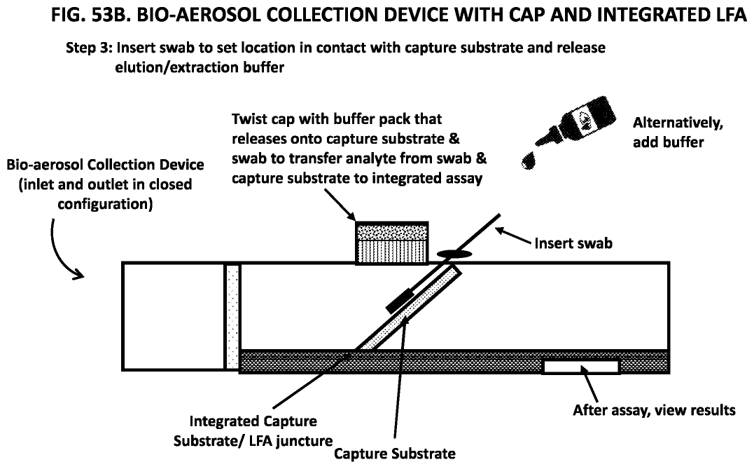

As illustrated in FIGS. 52 and 53, a user directs a bio-aerosol sample into a bio-aerosol collection device. The bio-aerosol sample can be provided through the user's mouth or nasal passage(s). A swab is also used to collect a nasopharyngeal (or oral) sample. As illustrated in FIG. 52, the swab sample is eluted using an appropriate buffer and the swab elution is then introduced to the bio-aerosol collection device to elute analyte from the capture substrate. The combined elution flows to the analytical device for analysis. As illustrated in FIG. 53B, the swab can be inserted into the bio-aerosol collection device. A buffer can be added from a buffer vial and/or via a capping system having a buffer pack as described herein. Close proximity of the swab and the capture substrate permit combination of the samples, which flow to a juncture with the analytical device for analysis.

Suitable bio-aerosol collection devices are described herein.

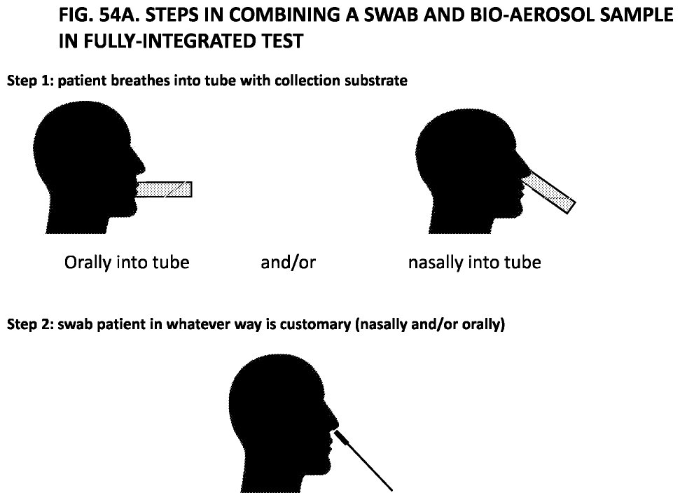

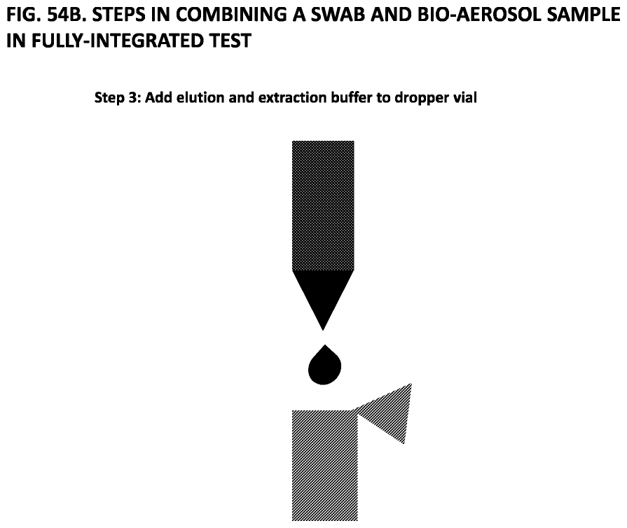

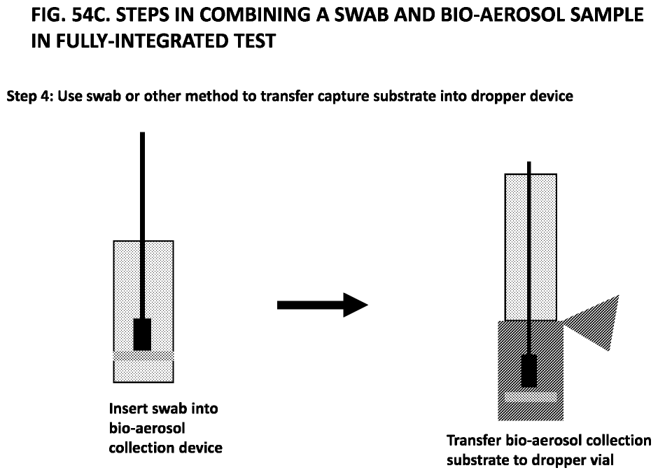

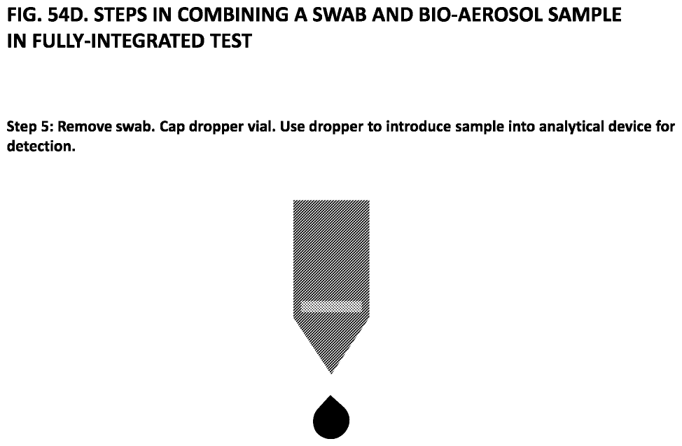

As illustrated in FIG. 54, bio-aerosol and swab samples are collected from a subject. An elution/extraction buffer is added to a dropper vial. The swab is used to transfer the capture substrate from the bio-aerosol collection device and into the dropper vial with the elution/extraction buffer. A drop of the combined sample is then introduced to an analytical device.

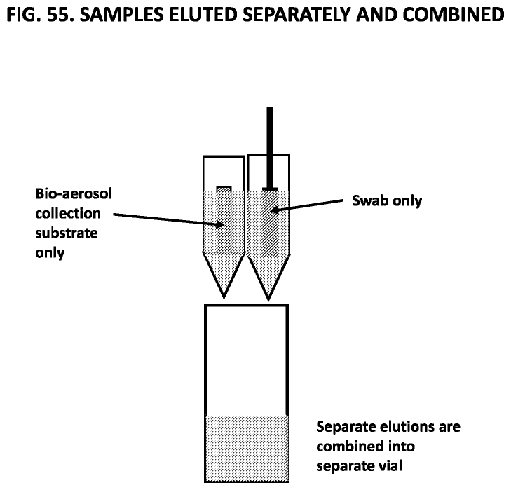

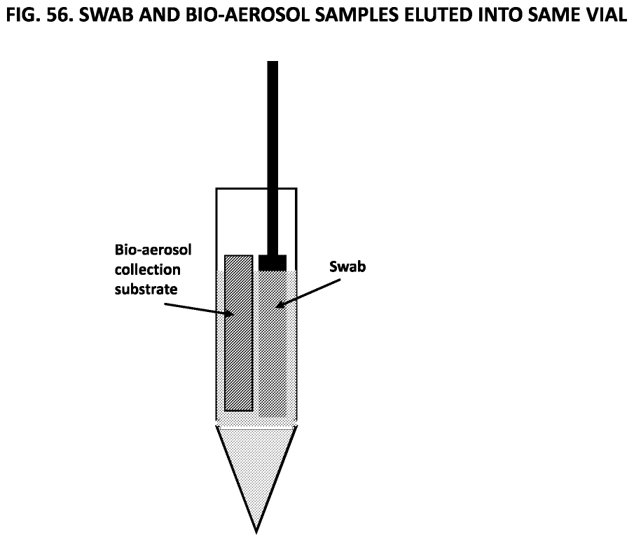

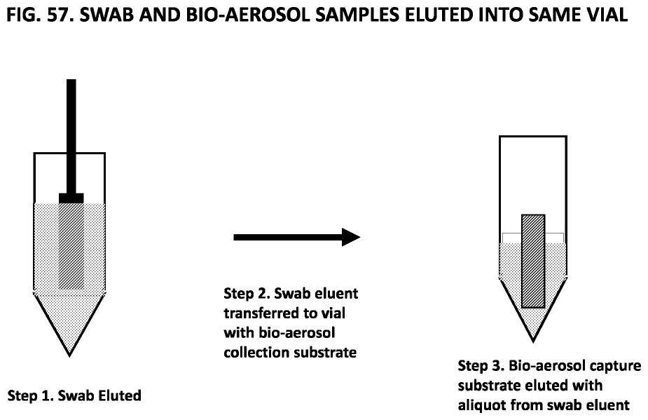

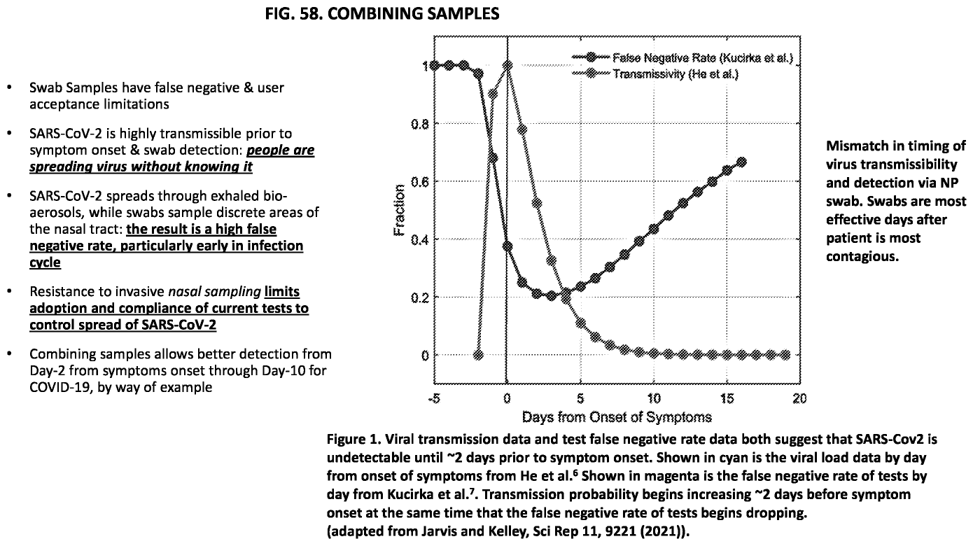

In another embodiment illustrated in FIG. 55, the bio-aerosol capture substrate and swab are eluted in separate vials of extraction/elution buffer, which are then combined to form a combined sample. In another embodiment illustrated in FIG. 56, the bio-aerosol capture substrate and swab are eluted in the same vial of extraction/elution buffer to form a combined sample. In another embodiment illustrated in FIG. 57, the swab is eluted in a vial of extraction/elution buffer and the swab is then the swab is discarded. The swab elution is then used to elute/extract the bio-aerosol capture substrate to form a combined sample.

The methods of the present disclosure combine samples obtained using a bio-aerosol collection device. In another embodiment, the methods of the present disclosure can combine a bio-aerosol sample and a first sample with a third sample collection method. Suitable third samples include, for example, sputum, nasal lavage, oral lavage, oral swab, and the like.

In another embodiment, the bio-aerosol sample eluate can be stored and/or transported to be analyzed at a laboratory test facility. Following collection, the sample can be placed in a sterile transport container containing a transport medium. Suitable transport media include, for example, viral transport medium (VTM), Amies transport medium, and sterile saline. The transport media can include, for example, RNase inhibitors, DNase inhibitors, protease inhibitors, preservatives, stabilizers, and other reagents for preserving the samples. Once placed in a transport container, the collected and combined samples can be stored until later analysis or processed for analysis. Processing can include lysis, extraction, and other known processing steps to analyze the combined sample for an analyte.

Any method for analyzing the samples of the present disclosure that are known in the art are suitable for analyzing the bio-aerosol sample collected using a bio-aerosol collection device as well as combined samples collected using a bio-aerosol collection device, swabs, lavage, aspirates, and combinations thereof. Suitable methods include, for example, polymerase chain reaction and other DNA and RNA amplification methods (e.g., PCR, RT-PCR, loop-mediated isothermal amplification (LAMP)), immunoassay detection methods such as, for example, Western blot analysis and enzyme linked immunosorbent analysis (ELISA), chromatography such as, for example, HPLC, gas chromatography, capillaripheresis, 2D and 3D gel electrophoresis, mass spectrometry, and combinations thereof.

### EXAMPLES

### Example 1

This Example outlines testing of different sample collection methods and combinations of sample collection methods.
1. Device:Blow tube
Use Case: General population
Detection Via: Multi-site (Oral+NP) Swab via PCR vs Bio-aerosol via PCR vs Sputum
Pathogens Tested: COVID+TB (need to determine single elution protocol)
Enrollment criteria: Presumed positive
Scientific Need: Compare rapid bio-aerosol collection vs NP swab collection method, ideally for days −2 to +7 of symptom onset for COVID. Sputum for TB

Behaviors for Bio-aerosol collection: Deep Breaths×20, Cough×10, Count to 20.
Collection time: 10-15 minutes

Sample Handling: POC worker removes bio-aerosol sample collector from device and places it in 5 mL Eppendorf. 1 mL of UTM. 10 sec mechanical vibration, spin for 30 to acquire sample in buffer.
2. Device:Blow tube
Use Case: General population
Detection Via: Multi-site (Oral+NP) Swab via PCR vs Bio-aerosol via PCR
Pathogens Tested: Respiratory panel
Enrollment criteria: Cough of any duration OR Shortness of breath OR Sore throat
Scientific Need: Compare rapid bio-aerosol collection vs NP swab collection method, ideally for days −2 to +7 of symptom onset for a panel of respiratory diseases

Behaviors for Bio-aerosol collection: Deep Breaths×20, Cough×10, Count to 20.
Collection time: 10-15 minutes

Sample Handling: POC worker removes bio-aerosol sample collector from device and places it in 5 mL Eppendorf. 1 mL of UTM. 10 sec mechanical vibration, spin for 30 to acquire sample in buffer.
3. Device: Blow tube
Use Case: General population
Detection Via: Multi-site (Oral+NS) Swab+Bio-aerosol via PCR vs NP swab via PCR
Pathogens Tested: COVID+TB
Enrollment criteria: Presumed positive
Scientific Need: Compare combining rapid bio-aerosol collection with NP swab collection method for days −2 to +7 of symptom onset for COVID
Behaviors for Bio-aerosol collection: Deep Breaths×20, Cough×10, Count to 20
Collection time: 10-15 minutes

Sample Handling: POC worker removes bio-aerosol sample collector from mask and places it in 5 mL Eppendorf. 1 mL of UTM. 10 sec mechanical vibration, spin for 30 to acquire sample in buffer.
4. Device: Blow tube
Use Case: General population
Detection Via: Multi-site (Oral+NS) Swab+Bio-aerosol via PCR vs NP swab via PCR
Pathogens Tested: Respiratory panel
Enrollment criteria: Cough of any duration OR Shortness of breath Or Sore throat

Scientific Need: Compare combining rapid bio-aerosol sample with NP swab collection method, ideally for days −2 to +7 of symptom onset for Respiratory panel.
Behaviors for Bio-aerosol collection: Deep Breaths×20, Cough×10, Count to 20
Collection time: 10-15 minutes

Sample Handling: POC worker removes bio-aerosol sample collector from mask and places it in 5 mL Eppendorf. 1 mL of UTM. 10 sec mechanical vibration, spin for 30 to acquire sample in buffer.
5. Device: Blow Tube attached to RDT
Use Case: General population
Detection Via: Multi-site (Oral+NP) Swab via PCR vs Bio-aerosol via RDT (visual and with reader)
Pathogens Tested: COVID
Enrollment criteria: Presumed positive

Scientific Need: Compare qualitative testing via RDT of rapid bio-aerosol sample only vs quantitative NP swab collection tested via reference PCR, ideally for days −2 to +7 of symptom onset for Respiratory panel.
Behaviors for Bio-aerosol collection: Deep Breaths×20, Cough×10, Count to 20
Collection time: 10-15 minutes
Sample Handling: POC worker attaches bio-aerosol collector to RDT. Patient performs behaviors. POC applied necessary elutin/extraction buffer drops to bio-aerosol substrate
6. Device: Blow Tube attached to RDT
Use Case: General population
Detection Via: Multi-site (Oral+NP) Swab via PCR vs NS swab+Bio-aerosol via RDT (visual and with reader)
Pathogens Tested: COVID
Enrollment criteria: Presumed positive

Scientific Need: Compare qualitative testing via RDT of rapid bio-aerosol sample combined with NS sample vs quantitative NP swab collection tested via reference PCR, ideally for days −2 to +7 of symptom onset for Respiratory panel.
Behaviors for Bio-aerosol collection: Deep Breaths×20, Cough×10, Count to 20
Collection time: 10-15 minutes

Sample Handling: POC worker attaches bio-aerosol collector to RDT. Patient performs bio-aerosol collection behaviors in device. POC worker swabs patient with an NS swab and elutes in RDT elution/extraction buffer as normal. POC applies necessary elutin/extraction buffer drops to bio-aerosol substrate to test via RDT.

While embodiments described herein include combining samples obtained using a bio-aerosol collection device with a sample collected using a swab collection method, it should be understood that the samples obtained using a bio-aerosol collection device can be analyzed alone (without combining with a second sample). Advantageously, the methods of the present disclosure make viral loads available in a density that can be used with a wide variety of tests such as amplification, antibody, antigen, and other paper-based tests. The methods of the present disclosure are particularly advantageous when a sample collected using a bio-aerosol collection device is combined with a swab sample because the combination of samples specifically increase viral load availability.

### Example 2

In this Example, nasal air samples collected using an exemplary embodiment of the sample collection device were analyzed for the presence of COVID.

Air samples were collected from patients who were presumed to be COVID positive. Patients also blew an air sample through their nostril into a collection device (“S” samples from Patient ID). Nasal swab samples (“B” samples from Patent ID) from each patient were also collected and analyzed.

Analyte collected in the collection substrate of the device was eluted with a buffer containing TX45. A TaqPath kit was used to perform PCR on each sample type to detect MS2, N Gene, ORF 1 ab, and S Genes.

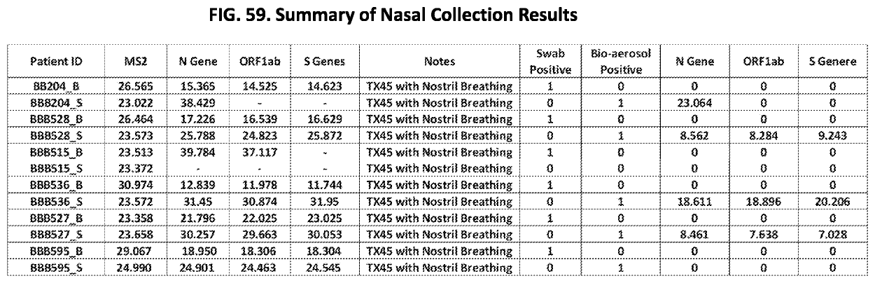

As summarized in the table depicted in FIG. 59, positive detection was obtained from nasal air samples. Surprisingly, detection in the nasal air sample was nearly 100% in concordance with samples collected using nasopharyngeal swabs.

This Example demonstrates that nasal air samples can be collected using the device and to detect analytes contained in the nasal air sample.

The methods of the present disclosure advantageously allow for overall sensitivity of testing and reducing false negative test results. In particular, combining an expiratory droplet sample with a swab sample can maximize test sensitivity and reduce the amounts of testing resources used to analyze the sample. Further, combining a bio-aerosol sample and a shed virus using a bio-aerosol collection device with other samples collected using different collection methods advantageously augments viral loads collected that can be tested thereby increasing test sensitivity. Combining a bio-aerosol sample and a shed virus collected using a bio-aerosol collection device with a sample collected using a swab collection method can also advantageously smooth out any signal diminishment inherent to sampling from the upper respiratory tract versus a saliva sample, for example. Combining bio-aerosol collection device with a sample collected using a swab collection can also advantageously assist with downstream test handling. Combining bio-aerosol collection device with a sample collected using a swab collection can also advantageously reduce false negatives because pathogen loads collected using at least two different sample collection methods can be increased.

1. A bio-aerosol collection device for collecting a bio-aerosol sample comprising:
a hollow housing comprising:
an inlet;
an outlet; and
a passage fluidly coupling the inlet and the outlet; and
a capture substrate disposed in the passage downstream of the inlet toward the outlet and configured to capture an analyte in the bio-aerosol sample.

2. The bio-aerosol collection device of claim 1, wherein the capture substrate is disposed between the inlet and the outlet.

3. The bio-aerosol collection device of claim 1, wherein the capture substrate extends through the outlet.

4. The bio-aerosol collection device of claim 1, further comprising a first closure configured to close the inlet and the outlet.

5. The bio-aerosol collection device of claim 1, further comprising a second closure configured to close the outlet.

6. The bio-aerosol collection device of claim 1, further comprising an analytical device reversibly coupled to the housing.

7. The bio-aerosol collection device of claim 1,
wherein the outlet is coupled to an analytical device; and
wherein at least a portion of the capture substrate contacts a sample region of the analytical device.

8. The bio-aerosol collection and analytical device of claim 7, wherein the capture substrate has a conical shape comprising a tip that contacts the sample region of the analytical device.

9. A bio-aerosol collection device for collecting a bio-aerosol sample, the device comprising:
a capture substrate configured to capture an analyte in the bio-aerosol sample;
a first bio-aerosol permeable protective layer disposed on a top surface of the capture substrate and covering the top surface of the capture substrate; and
a second bio-aerosol permeable protective layer disposed on a bottom surface of the capture substrate and covering the bottom surface of the capture substrate.

10. The bio-aerosol collection device of claim 9, further comprising a holder coupled to the capture substrate.

11. The bio-aerosol collection device of claim 9, further comprising a support layer comprising an opening sized and shaped to correspond to the size and shape of the capture substrate.

12. The bio-aerosol collection device of claim 11, wherein the support layer further comprises a holder coupled to the capture substrate, the support layer releasably connected to the holder, the holder configured to be disconnected from the support layer to remove the capture substrate from a remainder of the bio-aerosol collection device.

13. The bio-aerosol collection device of claim 12, wherein the holder is releasably coupled to the capture substrate.

14. A bio-aerosol collection device for collecting and analyzing a bio-aerosol sample, the device comprising:
a capture substrate configured to capture an analyte in the bio-aerosol sample;
a first compartment configured to cover a top surface of the capture substrate; and
a second compartment configured to cover a bottom surface of the capture substrate and comprising an analytical device wherein the analytical device comprises a sample pad in fluid contact with the bottom surface of the capture substrate;
wherein the first compartment and the second compartment are configured to be coupled together to form a seal.

15. The bio-aerosol collection device of claim 14, wherein the analytical device is selected from a lateral flow assay and a vertical flow assay.

16. The bio-aerosol collection device of claim 14, wherein the first compartment further comprises a buffer.

17. The bio-aerosol collection device of claim 16, wherein the first compartment comprises the buffer in a buffer pack.

18. The bio-aerosol collection device of claim 16, wherein the capture substrate is releasably coupled to a handle.

19. A method of collecting and analyzing a bio-aerosol sample, the method comprising: obtaining a bio-aerosol sample from a subject using a bio-aerosol collection device;
obtaining a swab sample from the subject;
combining the bio-aerosol sample and the swab sample; and
analyzing the combined sample.

20. The method of claim 19, wherein the bio-aerosol collection device is selected from
(a) a bio-aerosol collection device comprising:
a hollow housing comprising
an inlet;
an outlet; and
a passage fluidly coupling the inlet and the outlet; and
a capture substrate disposed in the passage downstream of the inlet toward the outlet and configured to capture an analyte in the bio-aerosol sample;
(b) a bio-aerosol collection device comprising:
a hollow housing comprising
an inlet;
an outlet coupled to an analytical device; and
a passage fluidly coupling the inlet and the outlet; and
a capture substrate disposed in the passage downstream of the inlet and configured to capture an analyte in the bio-aerosol sample wherein at least a portion of the capture substrate contacts a sample region of the analytical device;
(c) a bio-aerosol collection device comprising:
a capture substrate configured to capture an analyte in the bio-aerosol sample;
a first bio-aerosol permeable protective layer disposed on a top surface of the capture substrate and covering the top surface of the capture substrate;
a second bio-aerosol permeable protective layer disposed on a bottom surface of the capture substrate and covering the bottom surface of the capture substrate;
a handle coupled to the capture substrate; and
a support releasably connected to the handle, the handle configured to be disconnected from the support to remove the capture substrate from a remainder of the bio-aerosol collection device; and
(d) a bio-aerosol collection device comprising:
a capture substrate configured to capture an analyte in the bio-aerosol sample;
a first compartment configured to cover a top surface of the capture substrate;
a second compartment configured to cover a bottom surface of the capture substrate and comprising an analytical device wherein the analytical device comprises a sample pad in fluid contact with the bottom surface of the capture substrate;
wherein the first compartment and the second compartment are configured to be coupled together to form a seal.
